(19) United States
(12) Patent Application Publication
(10) Pub. No.: US 2021/0161894 A1
(43) Pub. Date: Jun. 3, 2021

(54) Compositions and Methods for Modulating Liver Endothelial Cell Fenestrations

(71) Applicants: David Le Couteur, Concord, New South Wales (AU); Victoria Cogger, Concord, New South Wales (AU)

(72) Inventors: David Le Couteur, Concord, New South Wales (AU); Victoria Cogger, Concord, New South Wales (AU)

(21) Appl. No.: 16/769,579

(22) PCT Filed: Dec. 4, 2018
(86) PCT No.: PCT/AU2018/000244

§ 371 Date: Jun. 3, 2020

(30) Foreign Application Priority Data
Dec. 4, 2017 (AU) 2017904879

(51) Int. Cl. A61K 31/506; A61K 31/519; A61K 31/4422; A61K 31/403; A61K 31/047; A61K 31/137; A61K 45/06; A61K 47/69

(52) U.S. Cl. CPC A61K 31/506; A61K 31/519; A61K 31/4422; A61K 47/6923; A61K 31/047; A61K 31/137; A61K 45/06; A61K 31/403

(57) ABSTRACT

There is provided compositions and methods for modulating the fenestration porosity, fenestration frequency or fenestration diameter of liver endothelial cells. In particular compositions comprising conjugates of quantum dots and a therapeutic are used to modulate the fenestration porosity, frequency or diameter of liver endothelial cells.

### TECHNICAL FIELD

The technology relates to the use of conjugates of a quantum dot and a therapeutic to modulate either or both of porosity and frequency of fenestrations in liver sinusoidal endothelial cells, for example in the treatment of age-related functional deterioration.

### RELATED APPLICATION

This application is based on and claims priority to Australian provisional patent application number 2017904879 filed by 4 Dec. 2017, the content of which is incorporated by reference in its entirety.

### BACKGROUND

There is an exponential increase in most diseases with old age, and consequently ageing is established as the most significant risk factor for disease. Approximately three quarters of people over 75 years have diabetes or pre-diabetes and/or hyperlipidaemia. These are established risk factors for cardiovascular outcomes and are also recognized as risk factors for geriatric conditions such as dementia, sarcopenia, frailty and osteoporosis.

The microcirculation of the liver has a unique morphology that facilitates the bi-directional exchange of substrates between hepatocytes and blood in the liver sinusoids. The cytoplasmic extensions of liver sinusoidal endothelial cells (LSECs) are very thin and perforated with transcellular pores known as fenestrations. Between 2-20% of the surface of the LSEC is covered by fenestrations and they are either scattered individually across the endothelial surface or clustered into groups called sieve plates. As there are no diaphragms or underlying basement membrane, fenestrations transform LSECs into a highly efficient ultrafiltration system, or ‘sieve’, which permits unimpeded transfer of dissolved and particulate substrates. Because of their extraordinary efficiency, fenestrations have minimal impact on substrate transfer in normal healthy livers.

As a person ages there is a consistent age-related functional deterioration in all the cells of the hepatic sinusoid including LSECs, stellate cells and Kupffer cells. Most notably, the LSECs in old age have markedly reduced porosity (% of LSEC surface area perforated by fenestrations) by about 50% with a similar 50% increase in the cross-sectional thickness of the LSEC. This age-related ‘pseudocapillarization’ is a feature of ageing and occurs without age-related pathology of hepatocytes or activation of stellate cells in mice, nonhuman primates and humans as well as prematurely in the transgenic Werner's syndrome (premature ageing) mouse.

The present inventors have observed that a number of drugs can be used to modulate either or both of porosity and frequency of fenestrations in liver sinusoidal endothelial cells. In addition the inventors have developed quantum dots that target liver sinusoidal endothelial cells and can be used for the targeted delivery of the drugs to liver sinusoidal endothelial cells.

### SUMMARY

In a first aspect there is provided a composition for modulating one or more of endothelial cell fenestration porosity, diameter and frequency in a subject, the composition comprising a therapeutic conjugate comprising a quantum dot and a therapeutic selected from an endothelin receptor antagonist, phosphodiesterase (PDE) inhibitor, calcium channel blocker, actin disruptor, lipid raft disruptor, 5-HT receptor agonist, TNF-related apoptosis-inducing ligand (TRAIL), nicotinamide adenine mononucleotide (NMN) or a combination thereof.

The quantum dot may be an Ag₂S, InP/ZnS or CuInS/ZnS quantum dot.

The subject may be an aged subject or a subject with an age related disease or condition.

The average diameter of the quantum dot may be about 2 nm, 3 nm, 4 nm, 5 nm, 6 nm, 7 nm, 8 mn, 9 nm, 10 nm, 11 nm, 12 nm, 13 nm, 14 nm, 15 nm, 16 nm, 17 nm, 18 nm or 20 nm. The therapeutic conjugate may be monodispersed.

The endothelin receptor antagonist may be selected from bosentan, sitaxentan, ambrisentan, atrasentan, zibotentan, macitentan, tezosentan, and edonentan.

The phosphodiesterase (PDE) inhibitor may be selected from sildenafil or its active analogues, tadalafil, vardenafil, udenafil, and avanafil.

The calcium channel blocker may be selected from amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine, fendiline. In another embodiment the calcium channel blocker is amlodipine.

The actin disruptor may be selected from cytochalasin, latrunculin, jasplakinolid, phalloidin, and swinholide.

The lipid raft disruptor may be selected from filipin, 7-ketocholesterol (7KC), and methyl-β-cyclodextrin.

The 5-HT receptor agonist may be selected from 2,5-Dimethoxy-4-iodoamphetamine (DOI), haloperidol, aripiprazole, asenapine, buspirone, vortioxetine, ziprasidone, methylphenidate, dihydroergotamine, ergotamine, methysergide, almotriptan, eletriptan, frovatriptan, naratriptan, rizatriptan, sumatriptan, zolmitriptan, yohimbine, lasmiditan, naratriptan, bufotenin, egonovine, lisuride, LSD, mescaline, myristicin, psilocin, psilocybin, fenfluramine, MDMA, norfenfluramine, methylphenidate, ergonovine, lorcaserin, tazodone, methyl-5-HT, qipazine, cinitapride, cisapride, dazopride, metoclopramide, mosapride, prucalopride, renzapride, tegaserod, zacopride, ergotamine, and valerenic acid.

In a second aspect there is provided a method of modulating one or more of endothelial cell fenestration, porosity, diameter and frequency in a subject, the method comprising administering to the subject an effective amount of a composition the first aspect.

The subject may be an aged subject or a subject with an age related disease or condition. The age related disease or condition may be selected from atherosclerosis, cardiovascular disease, arthritis, cataracts, age-related macular degeneration, hearing loss, osteoporosis, osteoarthritis, type 2 diabetes, hypertension, Parkinson's disease, dementia, Alzheimer's disease, age-related changes in the liver microcirculation, age-related dyslipidaemia, insulin resistance, fatty liver, liver fibrosis and liver cirrhosis.

The subject may be a subject with a disease or condition associated with one or more of reduced endothelial cell fenestration porosity, diameter and frequency.

The therapeutic or therapeutic conjugate may associate with an endothelial cell, for example the therapeutic conjugate may selectively associate with an endothelial cell. In some embodiments the endothelial cell is a liver endothelial cell.

The modulation may be an increase in one or more of endothelial cell fenestration porosity, diameter and frequency. For example, the increase may be at least 5%, 10%, 15%, 20%, 35%, 30%, 35%, 40%, 45%, 50%, 55%, 60%, 65%, 70%, 75%, 80%, 85%, 90%, 95%, or 100%.

In a third aspect there is provided use of a therapeutic conjugate comprising a quantum dot and a therapeutic for the manufacture of a medicament for modulating one or more of endothelial cell fenestration porosity, diameter and frequency in a subject.

In a fourth aspect there is provided a method of modulating one or more of endothelial cell fenestration porosity, diameter and frequency in a subject, the method comprising administering to the subject an effective amount of a phosphodiesterase (PDE) inhibitor, calcium channel blocker, actin disruptor, lipid raft disruptor, 5-HT receptor agonist, TNF-related apoptosis-inducing ligand (TRAIL), nicotinamide adenine mononucleotide (NMN) or a combination thereof.

The endothelin receptor antagonist may be selected from bosentan, sitaxentan, ambrisentan, atrasentan, zibotentan, macitentan, tezosentan, and edonentan.

The phosphodiesterase (PDE) inhibitor may be selected from sildenafil or its active analogues, tadalafil, vardenafil, udenafil, and avanafil.

The calcium channel blocker may be selected from amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine, fendiline. In another embodiment the calcium channel blocker is amlodipine.

The actin disruptor may be selected from cytochalasin, latrunculin, jasplakinolid, phalloidin, and swinholide.

The lipid raft disruptor may be selected from filipin, 7-ketocholesterol (7KC), and methyl-β-cyclodextrin.

The 5-HT receptor agonist may be selected from 2,5-Dimethoxy-4-iodoamphetamine (DOI), haloperidol, aripiprazole, asenapine, buspirone, vortioxetine, ziprasidone, methylphenidate, dihydroergotamine, ergotamine, methysergide, almotriptan, eletriptan, frovatriptan, naratriptan, rizatriptan, sumatriptan, zolmitriptan, yohimbine, lasmiditan, naratriptan, bufotenin, egonovine, lisuride, LSD, mescaline, myristicin, psilocin, psilocybin, fenfluramine, MDMA, norfenfluramine, methylphenidate, ergonovine, lorcaserin, tazodone, methyl-5-HT, qipazine, cinitapride, cisapride, dazopride, metoclopramide, mosapride, prucalopride, renzapride, tegaserod, zacopride, ergotamine, and valerenic acid.

In a fifth aspect there is provided use of a phosphodiesterase (PDE) inhibitor, calcium channel blocker, actin disruptor, lipid raft disruptor, 5-HT receptor agonist, TNF-related apoptosis-inducing ligand (TRAIL), nicotinamide adenine mononucleotide (NMN) or a combination thereof, for the manufacture of a medicament for modulating one or more of endothelial cell fenestration porosity, diameter and frequency in a subject.

### Definitions

The following are some definitions of terms used in the art that may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps, features, compositions and compounds.

The term ‘pharmaceutically acceptable salt’ refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, methanesulfonic, salicylic, p-hydroxybenzoic, phenylacetic, mandelic, ambonic, pamoic, pantothenic, sulfanilic, cyclohexylaminosulfonic, stearic, algenic, β-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, organic salts made from N,N′-dibenzylethylenediamine, chloroprocaine, choline, diethanolamine, ethylenediamine, meglumine (N methylglucamine), procaine, ammonium salts, quaternary salts such as tetramethylammonium salt, amino acid addition salts such as salts with glycine and arginine. In the case of compounds that are solids, it will be understood by those skilled in the art that the inventive compounds, agents and salts may exist in different crystalline or polymorphic forms, all of which are intended to be within the scope of the present invention and specified formulae.

The terms ‘treating’, ‘treatment’ and ‘therapy’ are used herein to refer to curative therapy, prophylactic therapy, palliative therapy and preventative therapy. Thus, in the context of the present disclosure the term ‘treating’ encompasses curing, ameliorating or tempering the severity of a medical condition or one or more of its associated symptoms.

The terms ‘therapeutically effective amount’ or ‘pharmacologically effective amount’ or ‘effective amount’ refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimising adverse side effects, including side effects typically associated with other therapies. Those skilled in the art can determine an effective dose using information and routine methods known in the art.

A ‘pharmaceutical carrier, diluent or excipient’ includes, but is not limited to, any physiological buffered (i.e., about pH 7.0 to 7.4) medium comprising a suitable water soluble organic carrier, conventional solvents, dispersion media, fillers, solid carriers, coatings, antibacterial and antifungal agents, isotonic and absorption delaying agents. Suitable water soluble organic carriers include, but are not limited to saline, dextrose, corn oil, dimethylsulfoxide, and gelatin capsules. Other conventional additives include lactose, mannitol, corn starch, potato starch, binders such as microcrystalline cellulose, cellulose derivatives such as hydroxypropylmethylcellulose, acacia, gelatins, disintegrators such as sodium carboxymethylcellulose, and lubricants such as talc or magnesium stearate.

‘Subject’ includes any human or non-human mammal. Thus, in addition to being useful for human treatment, the compounds of the present invention may also be useful for veterinary treatment of mammals, including companion animals and farm animals, such as, but not limited to dogs, cats, horses, cows, sheep, and pigs. In preferred embodiments the subject is a human.

In the context of this specification the term ‘administering’ and variations of that term including ‘administer’ and ‘administration’, includes contacting, applying, delivering or providing a therapeutic, QD, therapeutic-QD conjugate or composition to a subject by any appropriate means.

In the context of this specification the term ‘associates with’ refers to the arrangement of a therapeutic, QD or QD-conjugate with another element such as an LSEC to form a group. For example, the association of QD or QD-conjugate with an LSEC will occur when the QD or QD-conjugate contacts the LSEC or is internalized into the LSEC by endocytosis.

Throughout this specification, unless the context requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

### BRIEF DESCRIPTION OF THE DRAWINGS

FIG. 1. Effects drug treatments targeting NO dependent pathways on LSEC fenestrations and sieve plates in young and old mice. Scale bars are 1 μm.

FIG. 2. Effects of actin or lipid raft disruptors, death receptor promoters and nicotinamide mononucleotide on fenestrations in young and old mice. Scale bars are 1 μm.

FIG. 3. Effects of all drug treatments on fenestration porosity in (A) young and (B) old mice.

FIG. 4. Effects of all drug treatments on fenestration diameter in (A) young and (B) old mice.

FIG. 5. Effects of all drug treatments on fenestration frequency (number/μm²) in (A) young and (B) old mice.

FIG. 6. Percentage frequency of fenestration diameter histogram for control and NMN treated mice. Each data point represents the summation of 3390-4440 fenestration raw data points collected from n=2 mice.

FIG. 7. Transmission electron microscope image of Ag₂S Quantum dots. Scale bar is 200 nm.

FIG. 8. High resolution Transmission Electron Microscope images of Ag₂S QDs showing well-developed lattice in the box (A) and an average diameter of approximately 7 nm (B).

FIG. 9. Labelling of LSECs with of Ag₂S Quantum Dots after 15 minutes (A) and 1 hour (B) incubation. Scale bars are 500 nm.

FIG. 10. Labelling of liver sections with of Ag₂S Quantum Dots which can be seen as black dots. Scale bars are 500 nm.

FIG. 11. Effects of drug treatments on LSEC fenestration porosity and frequency, in young and old mice. (A) Sample SEM images of drug treatments in young mice. Scale bars of 1 μm are shown. Control image show fenestrations grouped in sieve plates (*). Bosentan, TRAIL, amlodipine, sildenafil and cytochalasin D treatments maintained sieve plates. (B) Changes in fenestration % porosity and (C) frequency (number per 1 μm2; grey bars) following drug treatments in young (white bars) and old (grey bars) mice.

FIG. 12. Effects of various drug treatments impacting LSEC fenestration diameter. (a) Changes in fenestration diameter induced by various drug treatments in young (white bars) and old (grey bars) mice. Drug treatments: simvastatin, bosentan, TRAIL, sildenafil, amlodipine, NMN, 7-ketocholesterol, cytochalasin D and DOI. All treatments were incubated at 37° C., 5% CO₂ for 30 mins using RPMI with or without dissolved drug. Manual counts of fenestration diameter were performed using SEM images at 10,000×. Data are presented relative to the % change compared to a control baseline. Each data point represents the average±SD of 8 images, using 616-3312 fenestration raw data points per treatment. All fenestrations <30 nm and gaps >300 nm were excluded from analysis. * Shows P<0.05 compared to young control; # shows P<0.05 compared to old control. Statistics were performed using Kruskal-Wallis with post-hoc Dunn's test to compare between groups, n=3 for all groups. (b) Sample SEM images of NMN and 7-ketocholesterol drug treatments in young and old mice. Scale bars of 1 μm are shown. Gaps (#) (>300 nm) were present in 7-ketocholesterol treatments. NMN treatments maintained sieve plates while 7-ketocholesterol treatments reduced lipid raft area. (c) Histogram of fenestration diameter in young control (white bars), old control (black bars), young NMN (light grey) old NMN (dark grey), young 7-ketocholesterol (light blue) and old 7-ketocholesterol (blue). Data are presented using the % frequency of diameters within the bin ranges shown.

FIG. 13. Correlations between porosity and frequency, cell viability and doses response curves relative to changes in porosity in young mice (a) Correlation plot between % porosity and frequency in young and old mice. Data shows all treatment data points (n=3 for each group; 20 groups). (b) Cell viability at a percentage relative to controls. Samples data was collected in triplicate with error bars showing SD (c) Dose concentration curves relative to changes in % porosity of fenestrations. Data for young mice are shown, drug concentrations are shown as a log function.

FIG. 14. Effects of drug treatments on the actin cytoskeleton, nitric oxide synthase and cyclic GMP. (A) dSTORM images showing actin cytoskeleton morphology changes promoted by various drug treatments in young mice. Images were produced following 40,000 image collections and processed using RapidSTORM software (45). Scale bar shown 5 μm, inserts showing gaps and individual fenestrations within actin. (B) Changes in actin densitometry induced by drug treatments in young mice. Data are presented as a bar graph (mean±SD) with the density of pixels per 1 μm². 8 images were captured using a dSTORM microscope (sample images shown in panel A); data analysis was performed using Image J software. Images were converted to binary data with measurements taken across the whole cell. (C) Changes in NOS densitometry induced by drug treatments in young mice. Data are presented as a bar graph (mean±SD) with the density of pixels per 1 μm². 5 images were captured using a dSTORM microscope; data analysis was performed using Image J software. Images were converted to binary data with measurements taken across the whole cell. * shows P<0.05 using Kruskal-Wallis with post-hoc Dunn's test to compare between groups, data were duplicated in a second mouse to confirm observation. (D) Intracellular cGMP, data are shown in pmol/10⁶ error bars show SD. Assay was performed in triplicate with a biological replicate. *** shows P<0.001 using Kruskal-Wallis with post-hoc Dunn's test to compare between groups. (E) Immunofluorescent images of LSECs stained for phosphorylated NOS (green) and NOS (red). Scale bar: 30 μm. Control, NMN and cytochalasin D demonstrated minimal staining, sildenafil treatment promoted co-localisation of phosphorated NOS and NOS (white arrows).

FIG. 15. Localization of Ag₂S Quantum dots to liver cells.

### DESCRIPTION OF EMBODIMENTS

Age-related pseudocapillarization of the liver sinusoidal endothelium contributes to dyslipidaemia and insulin resistance The healthy LSEC efficiently facilitates substrate transfer to the hepatocytes and so the role of the vasculature has usually been ignored in physiological models of hepatic function and clearance. Historically, the role of the LSEC in substrate transfer has been studied in liver cirrhosis and fibrosis where, with old age, there is loss of fenestrations (associated with other changes not seen in old age). Loss of fenestrations associated with liver disease causes reduced endothelial transfer and hepatic clearance of albumin, various drugs, bile salts and lipoproteins confirming that loss of fenestrations can influence substrate transfer.

Fenestrations have a diameter of 50-150 nm which allows passage of smaller lipoproteins including chylomicron remnants, while excluding larger particles such as chylomicrons and platelets. Old age is associated with impaired hepatic clearance of chylomicron remnants and its clinical manifestation of postprandial hypertriglyceridaemia. The latter is more closely associated with adverse cardiovascular and microvascular clinical outcomes.

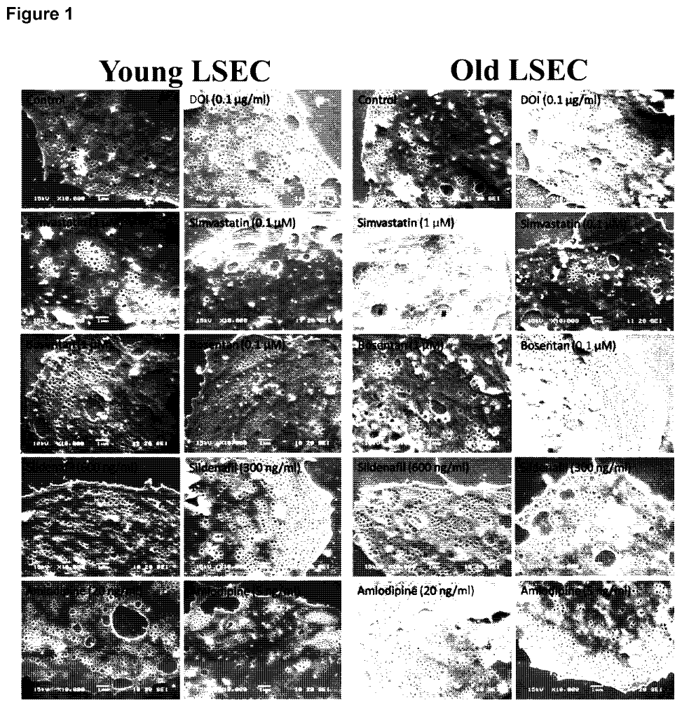

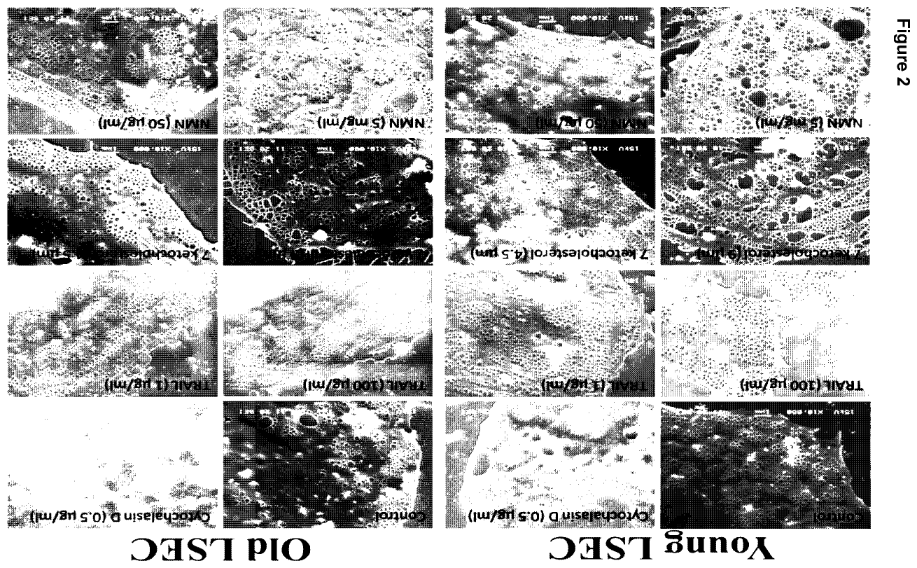

FIG. 1 shows one example of the age-related reduction in fenestrations and porosity of the LSEC cardiovascular outcomes in older people than the classical dyslipidemias. Using the multiple indicator dilution method in perfused rat livers, we showed that the transfer of lipoproteins (average diameter 53 nm) across the LSEC was almost totally abolished in livers from old animals. This provides a mechanism for age-related dyslipidemia and postprandial hyperlipidaemia which is accepted as a significant factor in age-related hyperlipidaemia. The inventors consider that strategies to maintain fenestration porosity into old age might ameliorate dyslipidemia and provide a means for the prevention of cardiovascular and microvascular disease in older people.

Old age is associated with insulin resistance and a markedly increased risk of diabetes. The multiple indicator dilution method in perfused livers has confirmed that insulin transfer across the LSEC is impaired in old age. Older rats show a significant reduction in the hepatic volume of insulin distribution, and this was consistent with the restriction of insulin to the vascular space. This was confirmed by whole animal insulin and glucose uptake studies showing reduced hepatic insulin uptake in old rats. Western blots and phosphor-proteomic analysis of livers also showed congruent reduced activation of the insulin receptor (IRS-1) and insulin pathways in old age. Measurements of glucose tolerance, homeostatic model assessment index (HOMA-IR), blood levels of insulin, C-peptide and glucagon showed that the reduced insulin action in the liver was associated with systemic impairment of insulin and glucose metabolism. These findings reveal fenestrations are crucial in hepatic insulin transfer consistent with other studies of hyper-fenestrated PDGF-B deficient mice. Increased fenestrations in these mice was associated with increased transendothelial transport, dramatically lower circulating insulin levels, increased insulin clearance and improved insulin sensitivity.

Together, these studies provide compelling evidence that fenestrations facilitate insulin transfer in the liver. Conversely, loss of fenestrations associated with age-related pseudocapillarization contributes to significant age-related risk factors for vascular disease—dyslipidemia and insulin resistance—by impairing the transfer of lipoproteins and insulin across the endothelium from sinusoidal blood to the hepatocytes and increasing fenestrations.

Acute loss of fenestrations, in the absence of other ageing changes, causes dyslipidaemia and insulin resistance Ageing is a complex process leading to impairment of many cellular pathways. To test the hypothesis that age-related loss of fenestrations contributes to dyslipidaemia and insulin resistance, the inventors aimed to evaluate the impact of acute defenestration in the absence of other ageing changes. This was tested using a surfactant, poloxamer 407 (P407) which was found to cause 30-80% loss of fenestrations within 24 hours of a single intraperitoneal injection. P407 administration caused a 10-fold increase in circulating lipoproteins, especially triglycerides and chylomicron remnants, while preventing the transfer of small chylomicrons across the LSEC. In more recent studies of insulin, it has been found that P407 prevented the passage of insulin across the LSEC leading to reduced phosphorylation of the insulin receptor substrate (IRS-1) with systemic insulin resistance (elevated HOMA-IR).These results affirm the key role of the fenestrations in human hepatic function and systemic health in ageing.

Fenestrations in the liver sinusoidal endothelium are regulated by lipid rafts In order to develop drugable targets for pharmacotherapies to maintain fenestrations into old age, we further investigated the proximate biological processes that regulate fenestrations and their density. The most potent known agents for increasing fenestrations are VEGF and various actin cytoskeleton disruptors which are linked because VEGF acts via its effects on the actin cytoskeleton.

Sieve plates containing fenestrations are intercalated between thickened areas of membrane (lipid rafts). 3D-SIM studies using the a lipid raft fluorescent stain (Bodipy FL C5 ganglioside GM1) found that there is a very strong inverse distribution between sieve plates and lipid rafts, with fenestrations and sieve plates only found in non-raft cell membrane.

As disclosed herein 7-ketocholesterol (which depletes lipid rafts) and/or cytochalasin D (an actin disruptor) increased fenestrations and decreased rafts, while Triton X-100 decreased fenestrations and increased rafts. Importantly, the effects of cytochalasin D on fenestrations were abrogated by co-administration of Triton X-100, proving that actin disruption increases fenestrations directly by its effects on membrane rafts. VEGF depleted lipid rafts and increased fenestrations.

The results are consistent with a sieve-raft interaction model, where fenestrations form in non-raft regions of endothelial cells once the membrane-stabilizing effects of actin cytoskeleton and membrane rafts are diminished.

While not wishing to be bound by any theory it is believed that the majority of agents that influence fenestrations act either via their effect on the actin cytoskeleton (VEGF, vasoactive agents such as bosentan and DOI (2,5-dimethoxy-4iodoamphetamine) or a direct effect on lipid rafts (7 ketocholesterol, TritonX100). Accordingly, lipid rafts or the regulation of lipid rafts by the actin cytoskeleton are targets for treatments that influence fenestrations.

A fundamental challenge in developing pharmacotherapies is targeting the active agent to the desired cell type or tissue. Fortunately, LSECs have unique properties that can be exploited as a drugable target. The LSEC is the most active and efficient endocytic cell in the body and is the main cell type responsible for the clearance of numerous blood-borne waste macromolecules (eg hyaluronan, immunoglobulins). The LSEC is densely populated with clathrin coated vesicles and numerous endocytotic receptors (eg mannose receptors, stabilin receptors, Fc gamma-receptor IIb2). This endocytic machinery is highly efficient in uptake and degradation of endogenous and exogenous waste material, including all major classes of biological macromolecules.

The inventors found that 7 nm CdTe/CdS (cadmium telurride/cadmium sulfide) quantum dots were almost exclusively taken up by LSECs within a few hours of administration. A major issue related to cadmium-based quantum dots, however, is their toxicity.

As disclosed herein silver chalcogenide-based quantum dots, which are considerably less toxic than CdTe/CdS quantum dots have been used to label or target LSEC. In particular, therapeutics that alter the porosity and frequency of fenestrations in LSECs can be conjugated to the silver chalcogenide-based quantum dots and targeted to the liver

In summary, the present inventors have established methods for altering age-related changes in lipoproteins and insulin activity related to age-related changes in the LSEC and its fenestrations by targeting LSECs using Ag₂S quantum dots alone or conjugated with a therapeutic. The inventors have also established that various unconjugated therapeutics are useful for modulating age-related changes in the LSEC fenestrations.

### Quantum Dots

Quantum dots (QDs) are small semiconductor particles, typically up to around 50 nm average diameter, that because of their small size have optical and electronic properties that differ from larger particles of the same material. A unique feature of LSECs is that QDs are taken up by the LSEC by endocytosis. Accordingly, any type of QD can be used in the methods and compositions described herein. For example the quantum dots may be core-type QDs, Core-Shell QDs, or alloyed QDs. In some embodiments the QDs are preferably non-toxic or have limited toxicity towards humans.

In some embodiments the QDs are free of heavy metals. For example the heavy metal free QDs may be Ag₂S, InP/ZnS (indium phosphide/zinc sulfide) or CuInS/ZnS (copper indium sulfide/zinc sulfide) QDs.

### Core-Type Quantum Dots

The quantum dots can be single component materials with uniform internal compositions, such as chalcogenides (selenides, sulfides or tellurides) of metals like cadmium, lead or zinc, for example, CdTe (cadmium telluride) or PbS (lead sulfide).

### Core-Shell Quantum Dots

The quantum dots can be core-shell QDs. The core-shell QDs can be prepared by any method known in the art. Such methods typically involve growing shells of a higher band gap semiconducting material around a core. For example a core-shell QD may have with CdSe in the core and ZnS in the shell. Coating quantum dots with shells improves quantum yield by passivizing nonradiative recombination sites and also makes them more robust to processing conditions. In some embodiments a non-toxic shell may be grown around a core that contains a toxic material.

### Alloyed Quantum Dots

The quantum dots can be alloyed QDs comprising a number of materials. Alloyed QDs are formed by alloying together two semiconductors with different band gap energies exhibited interesting properties distinct not only from the properties of their bulk counterparts but also from those of their parent semiconductors. For example, alloyed quantum dots of the compositions CdSₓSeᵢ₋ₓ/ZnS may be used in the methods and compositions described herein.

### Preparation of QDs

QDs may be provided or prepared for use in the compositions and methods described herein. Any method may be used to prepare QDs including colloidal synthesis, plasma synthesis, fabrication, and electrochemical assembly.

### Colloidal Synthesis

Colloidal synthesis involves heating a solution of precursor materials to a temperature high enough for the precursors to decompose to form monomers which then nucleate and generate nanocrystals. Temperature is an important factor in determining optimal conditions for QD formation and growth and the temperature needs to be high enough to allow rearrangement and annealing of atoms while allowing crystal growth. The concentration of monomers must also be controlled during crystal growth.

There are colloidal methods to produce QDs of lead sulfide, lead selenide, cadmium selenide, cadmium sulfide, cadmium telluride, indium arsenide, indium phosphide, silver sulphide and cadmium selenide sulfide. These QDs can contain as few as 100 to 100,000 atoms and have a diameter of about 1, 2, 3, 4, 5, 6, 7, 8, 9, or about 10 nm.

Large batches of QDs may be synthesized via colloidal synthesis thereby allowing QDs to be produced in amounts suitable for commercial applications.

### Plasma Synthesis

QDs can also be produced by known plasma techniques such as ion sputtering and plasma-enhanced chemical vapour deposition (PECVD). For example QDs of CuInSe₂, ZnO, Si, SiC, GaAs, GaSb, can be produced by ion-sputtering and QDs of Si, Ge, GaN, and InP can be produced by PECVD.

### Fabrication

QDs useful in the compositions and methods described herein can also be produced by self-assembly. In some embodiments such QDs have an average diameter of about 5 nm to about 50 nm. In some embodiment the QDs can be defined by lithographically patterned gate electrodes, or by etching on two-dimensional electron gasses in semiconductor heterostructures.

In some embodiments the QDs may self-assemble. For example the QD can nucleate spontaneously under certain conditions during molecular beam epitaxy (MBE) and metallorganic vapor phase epitaxy (MOVPE) when a material is grown on a substrate to which it is not lattice matched. The resulting strain produces coherently strained islands on top of a two-dimensional wetting layer. The islands can be subsequently buried to form the quantum dot.

Individual quantum dots can be created from two-dimensional electron or hole gases present in remotely doped quantum wells or semiconductor heterostructures called lateral quantum dots. The sample surface is coated with a thin layer of resist. A lateral pattern is then defined in the resist by electron beam lithography. This pattern can then be transferred to the electron or hole gas by etching, or by depositing metal electrodes that allow the application of external.

### Electrochemical Assembly

Ordered arrays of QDs may be self-assembled by electrochemical techniques. In these methods a template is created by causing an ionic reaction at an electrolyte-metal interface which results in the spontaneous assembly of nanostructures, including quantum dots, onto the metal which is then used as a mask for mesa-etching the nanostructures on a chosen substrate.

QDs produced by any of the above methods can also be coated or passivated by a non-toxic compound. For example a lead sulfide QD may be passivated at least one of oleic acid, oleyl amine and hydroxyl ligands. Passivation can also be used to provide a group that can bind a therapeutic in order to generate the QD-conjugates described herein.

### Silver Sulfide (Ag₂S) Quantum Dots

Silver sulfide (Ag₂S) quantum dots have low or no toxicity to mammals and may also have near-infrared fluorescence. Ag₂S quantum dots are hydrophobic and should be functionalized (i.e. transformed from hydrophobic form into hydrophilic) to be useful for methods of treatment or for conjugating with a therapeutic. Ag₂S quantum dots have a superlattice structure that is difficult to modify.

The QDs can be prepared in a two-step process comprising 1) preparing hydrophobic silver sulfide quantum dots from a silver source and a long chain thiol; and 2) functionalising the quantum dots with an equivalent or excessive amount of an organosulfur compound, a thiol or a mercapto-containing hydrophilic reagent in polar organic solvent, so that the surface of the silver sulfide quantum dots is attached with hydrophilic groups.

The silver source and the long chain thiol are reacted to obtain hydrophobic silver sulfide quantum dots. Then the surface functionalization of the hydrophobic silver sulfide quantum dots as prepared is conducted with an sulfur containing hydrophilic reagent.

### Preparation of Hydrophobic Silver Sulfide Quantum Dots

Preparation of the hydrophobic silver sulfide quantum dots comprises the following steps:
1) heating a mixed reaction system containing a silver source and a long chain thiol to a reaction temperature of 50-400° C. in a closed environment to react for a reaction time of about 1-10 or more hours; and
2) cooling the mixed reaction system to room temperature, then adding a polar solvent, centrifuging and washing to obtain the hydrophobic quantum dots;

The silver source may be one or more of diethyldithiocarbamate, silver nitrate, silver diethyldithiocarbamate, silver dihydrocarbyldithiophosphate, dioctyl silver sulfosuccinate, silver thiobenzoate, silver acetate, silver dodecanoate, silver tetradecanoate and silver octadecanoate.

The long chain thiol may be one or more of octanethiol, undecanethiol, dodecanethiol, tridecanethiol, tetradecanethiol, pentadecanethiol, hexadecanethiol, octadecanethiol, eicosanethiol, hexanethiol, 1,6-hexanedithiol, and 1,8-octanedithiol.

The reaction temperature may be about 50, 75, 100, 125, 150, 175, 200, 225, 250, 275, 300, 325, 350, 375, or about 400° C.

The mixed reaction system may be heated to the reaction temperature at a rate of about 5-50° C./min. For example the heating rate may be about 5, 10, 15, 20, 25, 30, 35, 40, 45 or 50° C./min.

The polar solvent added in step 2 may be any one of ethanol, methanol, acetone and 1-methyl-2-pyrrolidone or any combination thereof.

In one embodiment oxygen is substantially removed from the mixed reaction system before heating. This may be achieved for example by placing the reacting system under a vacuum, purging with nitrogen or other gas, or a combination of both. In one embodiment the mixed reaction system is maintained under nitrogen or other gas for the reaction time.

The reaction time may be about 1, 2, 3, 4, 5, 6, 7, 8, 9, or 10 or more hours.

The hydrophobic Ag₂S quantum dots prepared by the method described herein have monoclinic structure and an average diameter of about 2 nm, 3 nm, 4 nm, 5 nm, 6 nm, 7 nm, 8 mn, 9 nm or 10 nm.

### Functionalisation

The Ag₂S QDs disclosed herein can be functionalised with hydrophilic groups attached to the surface thereof. The hydrophilic groups are derived from a mercapto or thiol-containing hydrophilic reagent or an organosulfur compound such as a-lipoic acid (thioctic acid), cysteine, or methionine. The hydrophilic reagent may be a mercapto-containing hydrophilic reagent such as mercaptoacetic acid, mercaptopropionic acid, cysteine, cysteamine, thioctic acid and ammonium mercaptoacetate or any combination thereof. In another embodiment the hydrophilic reagent may be a thiol-containing hydrophilic reagent such as an alkanethiol. The alkanethiol may be octanethiol, dodecanethiol, tert-dodecanethiol, eicosanethiol or any combination thereof. In another embodiment the hydrophilic agent may be any combination of an organosulfur compound, a mercapto and a thiol-containing hydrophilic reagent. In another embodiment the hydrophilic agent is thioctic acid.

In one embodiment the mole number of the hydrophilic reagent is more than or equal to that of the hydrophobic silver sulfide quantum dots. The ratio of the mole number of the hydrophilic reagent to that the hydrophobic silver sulfide quantum dots can be adjusted depending on the actual requirement during the preparation process.

Functionalisation occurs in a polar solvent. For example the polar organic solvent may be any one of cyclohexane, ethanol, methanol, acetone and 1-methyl-2-pyrrolidone or any combination thereof.

In one embodiment the hydrophobic QDs are dispersed in the polar organic solvent and the hydrophilic reagent is added and this mixed system allowed to react at about 1-80° C. for about 1, 2, 3, 4, 5, 6, 7, 8, 9, or 10 or more hours.

The reaction temperature may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 ,31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80° C.

In some embodiments the mixed system may be continuously or intermittently sonicated during the reaction.

The functionalised Ag₂S QDs prepared by the method described herein are monodispersed, do not aggregate, are hydrophilic, stable and can be used for labelling or targeting liver cells. In particular, and with reference to Example 7 Ag₂S QDs target the liver, in particle the LSECs. In some embodiments the Ag₂S QDs specifically label the LSECs.

### Therapeutic Coniuqates

Some compounds such as 7-ketocholesterol and Cytochalasin D are known to increase the porosity of LSEC fenestrations. In addition, other therapeutics such as sildenafil and amlodipine are demonstrated herein to also modulate at least one of fenestration porosity, frequency and diameter. In the context of treating age related disease or aging by modifying LSEC fenestrations the systemic administration of such compounds may be associated with unnecessary or unwanted therapeutic effects. Accordingly, it is advantageous to target the therapeutic the LSEC using a conjugate of the therapeutic and a Ag₂S QD to avoid unnecessary or unwanted therapeutic side-effects.

Standard conjugation chemistry may be used for conjugation of the functionalised Ag₂S QDs to a therapeutic. Preparation of a therapeutic-QD conjugate includes the steps of providing a QD, providing a coupling agent, providing a therapeutic or derivative thereof and incubating the mixture to form a crude therapeutic-QD conjugate. Alternatively the functionalised Ag₂S QDs may be reacted with a coupling agent before the addition of the therapeutic.

Crude therapeutic-QD conjugate may then be purified for example by filtration or centrifugation to obtain a therapeutic-QD conjugate suitable for used in the methods described herein.

In some embodiments the therapeutic is conjugated directly to hydrophobic Ag₂S QD. In other embodiments the therapeutic is conjugated to the functionalised Ag₂S QDs via the organic layer that is used to render the QDs hydrophilic, biocompatible or both.

The therapeutic can be conjugated to the functionalised Ag₂S QDs via an amide or an ester linkage. However it should be understood that other bonds may be formed (e.g., both covalent and non-covalent). In one embodiment, the therapeutic is conjugated to the functionalised Ag₂S QDs either covalently, physically, ion pairing, or Van der Waals' interactions. The bond may be formed by an amide, ester, thioester, or thiol group.

Standard conditions for conjugating the therapeutic to the functionalised Ag₂S QDs can be employed. For example, the conjugation (of the functionalised Ag₂S QDs to a coupling agent or the coupling of the functionalised Ag₂S QDs with a coupling agent and a therapeutic) may occur in a buffered solution over a time from about 5 minutes to about 12 hours. For example the coupling may occur over a time of about 5 minutes, 10 minutes, 20 minutes, 30 minutes, 40 minutes, 50 minutes, 1 hour, 2, hours, 3 hours, 4 hours, 5 hours, 6hours, 7 hours, 8 hours, 9 hours or about 10 hours. The temperature of the coupling conditions may be from about 1° C. to about 100° C. For example the temperature may be 1, 2, 3, 4, 5, 6, 7, 8, 9, 10, 11, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or about 100° C.

The conjugation conditions may be constant or varied during the reaction. For example the reaction may be performed at a constant temperature or the temperature may be varied throughout the reaction or the reaction may proceed with stepwise changes in the one or more conditions.

Coupling agents may be used to form an amide or an ester group between the carboxyl functions on the QDs and either the carboxyl or the amine end groups on the therapeutic. Linkers or coupling agents may include benzotriazolyloxy-tris(dimethylamino) phosphonium hexafluorophosphate (BOP) and carbodiim ides such as dicyclohexylcarbodiimide (DCC), diisopropylcarbodiimide (DIC), 1-(3-dimethyl-aminopropyl)-3-ethylcarbodiimide hydrochloride (EDC), N-hydroxysuccinamide, and sulfo-N-hydroxysuccinamide (NHS).

In one embodiment the coupling agent is NHC, EDC or both.

In one embodiment the quantum dot bearing a carboxyl end group and a therapeutic may be mixed in a solvent. A coupling agent, such as NHS, may be added to the mixture. The reaction mixture may be incubated at elevated temperatures. The crude therapeutic-QD conjugate may be subject to purification to obtain a therapeutic-QD conjugate that may be used in the formulations and methods herein.

Standard solid state purification methods may be used to separate the therapeutic-QD conjugates from unused reagents. For example several cycles of filtering and washing with a suitable solvent may be necessary to remove excess unreacted therapeutic and NHS. Alternatively or in addition the therapeutic-QD conjugates may be sedimented by centrifugation and resuspended in a suitable solvent.

Suitable solvents include any biocompatible liquid such as water or buffered saline e.g. phosphate buffered saline.

### Therapeutics

Any therapeutic may be conjugated to the hydrophobic Ag₂S QDs or functionalised Ag₂S QDs.

The therapeutic can be an endothelin receptor antagonist. For example the endothelin receptor antagonist may be selected from the group comprising bosentan (Tracleer®), sitaxentan, ambrisentan, atrasentan, BQ-123, zibotentan, macitentan, tezosentan, BQ-788, 192621 and edonentan. In one embodiment the endothelin receptor antagonist is bosentan.

The therapeutic can be a phosphodiesterase (PDE) inhibitor. For example the PDE inhibitor may selected from the group consisting of aminophylline, IBMX (3-isobutyl-1-methylxanthine), paraxanthine, pentoxifylline, theobromine, theophylline, a methylated xanthine, vinpocetine, EHNA (erythro-9-(2-hydroxy-3-nonyl)adenine), BAY 60-7550 (2-[(3,4-dimethoxyphenyl)methyl]-7-[(1R)-1-hydroxyethyl]-4-phenylbutyl]-5-methyl-imidazo[5,1-f][1,2,4]triazin-4(1H)-one), oxindole, PDP (9-(6-Phenyl-2-oxohex-3-yl)-2-(3,4-dimethoxybenzyl)-purin-6-one), inamrinone, milrinone, enoximone, anagrelide, cilostazol, pimobendan, mesembrenone, rolipram, ibudilast, piclamilast, luteolin, drotaverine, roflumilast, apremilast, crisaborole, sildenafil, active analogues of sildenafil, tadalafil, vardenafil, udenafil avanafil, dipyridamole, icariin, 4-Methylpiperazine, and pyrazolo pyrimidin-7-1, and papaverine.

In one embodiment the PDE inhibitor is one or more of sildenafil, tadalafil, vardenafil, udenafil avanafil. In another embodiment the PDE inhibitor is sildenafil.

The therapeutic can be a calcium channel blocker. For example the calcium channel blocker may be selected from the group comprising amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine, fendiline, gallopamil, verapamil, diltiazem, mibefradil, bepridil, flunarizine, and fluspirilene.

In one embodiment the calcium channel blocker may be selected from the group comprising amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine, fendiline. In another embodiment the calcium channel blocker is amlodipine.

The therapeutic can be an actin disruptor or a lipid raft disruptor. Examples of suitable actin disruptors are a cytochalasin, latrunculin, jasplakinolid, phalloidin, swinholide. In some embodiments the cytochalasin is selected from cytochalasin A, B, C, D, E, F, H, G, J or any combination thereof. In one embodiment the cytochalasin is cytochalasin D.

Examples of suitable lipid raft disruptors are filipin, 7-ketocholesterol (7KC), methyl-β-cyclodextrin.

Other suitable therapeutics include TNF-related apoptosis-inducing ligand (TRAIL) and nicotinamide adenine mononucleotide (NMN).

The therapeutic can be a 5-HT receptor agonist. For example the 5-HT receptor agonist therapeutic may be selected from the group comprising 2,5-Dimethoxy-4-iodoamphetamine (DOI), vilazodone (viibryd), flesinoxan, gepirone, haloperidol, ipsapirone, quetiapine, trazodone, yohimbine, tandospirone, aripiprazole, asenapine, buspirone, vortioxetine, ziprasidone, methylphenidate, dihydroergotamine, ergotamine, methysergide, almotriptan, eletriptan, frovatriptan, naratriptan, rizatriptan, sumatriptan, zolmitriptan, yohimbine, lasmiditan, naratriptan, bufotenin, egonovine, lisuride, LSD, mescaline, myristicin, psilocin, psilocybin, fenfluramine, MDMA, norfenfluramine, methylphenidate, ergonovine, lorcaserin, tazodone, methyl-5-HT, qipazine, cinitapride, cisapride, dazopride, metoclopramide, mosapride, prucalopride, renzapride, tegaserod, zacopride, ergotamine, and valerenic acid.

### Therapeutic Use

There are significant age-related changes in the liver endothelium. For example, the microcirculation of the liver has a unique morphology that facilitates the bi-directional exchange of substrates between hepatocytes and blood in the liver sinusoids. The cytoplasmic extensions of liver sinusoidal endothelial cells (LSECs) are very thin and perforated with transcellular pores known as fenestrations. Between 2-20% of the surface of the LSEC is covered by fenestrations and they are either scattered individually across the endothelial surface or clustered into groups called sieve plates. As there are no diaphragms or underlying basement membrane, fenestrations transform LSECs into a highly efficient ultrafiltration system, hence a ‘sieve’, which permits unimpeded transfer of dissolved and particulate substrates within a size threshold. Because of their extraordinary efficiency, fenestrations have minimal impact on substrate transfer in normal healthy livers.

There is dramatic but consistent age-related functional deterioration and structural changes in all the cells of the hepatic sinusoid: LSECs, stellate cells and Kupffer cells (Le Couteur, D G, et al. 2008. Old age and the hepatic sinusoid. Anat Rec (Hoboken) 291: 672-83). Most notably, the LSECs in old age had markedly reduced porosity (% of LSEC surface area perforated by fenestrations) by about 50% with a similar 50% increase in the cross-sectional thickness of the LSEC. These morphological changes were accompanied by altered expression of many vascular proteins including von Willebrands factor, ICAM-1, laminin, caveolin-1 and various collagens. This age-related ‘pseudocapillarization’ is a feature of ageing in rats, mice, nonhuman primates and humans, as well as prematurely in the transgenic Werner's syndrome (premature ageing) mouse.

The QDs, conjugates or compositions thereof can be administered to a subject to modulate one or more of fenestration porosity, diameter and frequency in endothelial cells, particularly liver sinusoidal endothelial cells(LSECs). Accordingly, in one embodiment there is provided a method of modulating one or more of fenestration porosity, diameter and frequency.

In one embodiment there is provided a method of treatment of a disease or condition associated with one or more of reduced LSEC one or more of fenestration porosity, diameter and frequency, the method comprising administering to the subject an effective amount of a Ag₂S QD-therapeutic conjugate, or a composition thereof.

In some embodiments the subject is a human.

In some embodiments the subject is suffering from an age related disease or condition.

An age related disease is any disease or condition is most often seen with increasing frequency with increasing age and may include consequences of the aging process such as functional decline of one or more organs. Examples of age related diseases include atherosclerosis, cardiovascular disease, arthritis, cataracts, Age-related macular degeneration, hearing loss, osteoporosis, osteoarthritis, type 2 diabetes, hypertension, Parkinson's disease, dementia, Alzheimer's disease, age-related changes in the liver microcirculation, age-related dyslipidaemia, insulin resistance, fatty liver, liver fibrosis, and liver cirrhosis.

The QDs, therapeutics and therapeutic conjugates described herein may be administered as a formulation comprising a pharmaceutically effective amount of the compound in association with one or more pharmaceutically acceptable excipients including carriers, vehicles and diluents. The term ‘excipient’ herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solid dosage form such as a tablet, capsule, or a solution or suspension suitable for oral, parenteral, intradermal, subcutaneous, or topical application. Excipients can include, by way of illustration and not limitation, diluents, disintegrants, binding agents, adhesives, wetting agents, polymers, lubricants, glidants, stabilizers, and substances added to mask or counteract a disagreeable taste or odor, flavors, dyes, fragrances, and substances added to improve appearance of the composition. Acceptable excipients include (but are not limited to) stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutically acceptable materials. Examples of excipients and their use is described in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

The QDs, therapeutics and therapeutic conjugates described herein may be formulated for oral, injectable, rectal, parenteral, subcutaneous, intravenous or intramuscular delivery. Non-limiting examples of particular formulation types include tablets, capsules, caplets, powders, granules, injectables, ampoules, vials, ready-to-use solutions or suspensions, lyophilized materials, suppositories and implants. The solid formulations such as the tablets or capsules may contain any number of suitable pharmaceutically acceptable excipients or carriers described above. The conjugates may also be formulated for sustained delivery.

Tablets and capsules for oral administration may be in unit dose presentation form, and may contain conventional excipients such as binding agents, for example, acacia, gelatin, sorbitol, tragacanth, or polyvinylpyrollidone; fillers, for example lactose, sugar, maize-starch, calcium phosphate, sorbitol or glycine; tabletting lubricants, for example, magnesium stearate, talc, polyethylene glycol or silica; disintegrants, for example, potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example, sorbitol, methyl cellulose, glucose syrup, gelatin, hydroxyethyl cellulose, carboxymethyl cellulose, aluminium stearate gel or hydrogenated edible fats, emulsifying agents, for example, lecithin, sorbitan monooleate, or acacia; non-aqueous vehicles (which may include edible oils), for example, almond oil, oily esters such as glycerin, propylene glycol, or ethyl alcohol; preservatives, for example, methyl or propyl p-hydroxybenzoate or sorbic acid; and, if desired, conventional flavouring or colouring agents.

For parenteral administration, including intravenous, intramuscular, subcutaneous, or intraperitoneal administration, fluid unit dosage forms may be prepared by combining the QDs, conjugates and/or a therapeutic with a sterile vehicle, typically a sterile aqueous solution which is preferably isotonic with the blood of the subject. Depending on the vehicle and concentration used, the therapeutic or conjugate may be either suspended or dissolved in the vehicle or other suitable solvent. In preparing solutions, the therapeutic or conjugate may be dissolved in water for injection and filter sterilized before filling into a suitable vial or ampoule and sealing. Advantageously, agents such as a local anaesthetic, preservative and buffering agents can be dissolved in the vehicle. To enhance the stability, the composition may be frozen after filling into the vial and the water removed under vacuum. The dry lyophilized powder may then be sealed in the vial and an accompanying vial of water for injection may be supplied to reconstitute the liquid prior to use. Parenteral suspensions are prepared in substantially the same manner except that the conjugates are suspended in the vehicle instead of being dissolved and sterilization cannot be accomplished by filtration. The conjugates can be sterilized by exposure to ethylene oxide before suspending in the sterile vehicle. A surfactant or wetting agent may be included in the composition to facilitate uniform distribution of the compound.

The therapeutics, QD or QD-therapeutic conjugate can be administered topically or by transdermal routes, for example by using transdermal skin patches. In some embodiments transdermal administration is used to achieve a continuous dosage throughout the dosage regimen. Suitable transdermal formulations may be prepared by incorporating the therapeutic, QD or QD-therapeutic conjugate in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a subject.

The amount of therapeutically effective therapeutic or conjugate that is administered and the dosage regimen for treating a disease condition with the conjugates and/or pharmaceutical compositions of the present invention depends on a variety of factors, including the age, weight, sex, and medical condition of the subject, the severity of the disease, the route and frequency of administration, the particular conjugates employed, as well as the pharmacokinetic properties (eg, adsorption, distribution, metabolism, excretion) of the individual treated, and thus may vary widely. Such treatments may be administered as often as necessary and for the period of time judged necessary by the treating physician. One of skill in the art will appreciate that the dosage regime or therapeutically effective amount of the compound to be administrated may need to be optimized for each individual.

A composition may contain the therapeutic or conjugate in the range of about 0.1 mg to 2000 mg, typically in the range of about 0.5 mg to 500 mg and more typically between about 1 mg and 200 mg. A daily dose of about 0.01 mg/kg to 100 mg/kg body weight, typically between about 0.1 mg/kg and about 50 mg/kg body weight, may be appropriate, depending on the route and frequency of administration. The daily dose will typically be administered in one or multiple, e.g., two, three or four, doses per day.

As set out above there is provided a method of modulating one or more of fenestration porosity, diameter and frequency in endothelial cells, particularly liver sinusoidal endothelial cells(LSECs), by the administration of the therapeutic or conjugates described herein

In one embodiment the methods disclosed herein increase the porosity of fenestrations in endothelial cells, such as LSECs by 5%, 10%, 15%, 20%, 35%, 30%, 35%, 40%, 45%, 50%, 55%, 60%, 65%, 70%, 75%, 80%, 85%, 90%, 95%, or at least 100% compared to the average porosity prior to treatment.

In another embodiment increase fenestration frequency of fenestrations in endothelial cells, such as LSECs by 5%, 10%, 15%, 20%, 35%, 30%, 35%, 40%, 45%, 50%, 55%, 60%, 65%, 70%, 75%, 80%, 85%, 90%, 95%, or at least 100% compared to the average fenestration frequency prior to treatment.

In one embodiment increase the average diameter of fenestrations in endothelial cells, such as LSECs by 5%, 10%, 15%, 20%, 35%, 30%, 35%, 40%, 45%, 50%, 55%, 60%, 65%, 70%, 75%, 80%, 85%, 90%, 95%, or at least 100% compared to the average fenestration diameter prior to treatment.

In some embodiments at least one of fenestration porosity, diameter and frequency in an aged subject are returned to or maintained at levels seen in a healthy non-aged subject.

An aged subject is a subject that is 45 years old or older. In some embodiments an aged subject is 40 years old or older.

The therapeutics or conjugates described herein may be administered along with a pharmaceutical carrier, diluent or excipient as described above. Alternatively, or in addition, the therapeutics or conjugates may be administered in combination with other agents, for example, other therapeutic agents.

The terms ‘combination therapy’ or ‘adjunct therapy’ in defining use of a therapeutic or therapeutic conjugate described herein and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments more conjugates may be formulated or administered in combination with one or more other therapeutic agents. Thus, in some embodiments, one or more conjugates may be included in combination treatment regimens with other known treatments or therapeutic agents, and/or adjuvant or prophylactic agents.

A number of agents are available in commercial use, in clinical evaluation and in pre-clinical development, which could be selected for treatment of aging or of an age related disease.

Suitable agents which may be used in combination therapy will be recognized by those of skill in the art. Suitable agents are listed, for example, in the Merck Index, An Encyclopaedia of Chemicals, Drugs and Biologicals, 12th Ed., 1996, the entire contents of which are incorporated herein by reference.

For example, when used in the treatment of age related diseases, or other diseases with loss of fenestrations the therapeutic conjugates or therapeutics described herein may be administered with an additional agents.

Combination regimens may involve the active agents being administered together, sequentially, or spaced apart as appropriate in each case. Combinations of active agents including the QDs and conjugates described herein may be synergistic.

The co-administration of the QDs or conjugates described herein may be effected by the QDs or conjugates being in the same unit dose as another active agent, or the QDs or conjugates and one or more other active agent(s) may be present in individual and discrete unit doses administered at the same, or at a similar time, or at different times according to a dosing regimen or schedule. Sequential administration may be in any order as required, and may require an ongoing physiological effect of the first or initial compound to be current when the second or later compound is administered, especially where a cumulative or synergistic effect is desired.

Embodiments of the invention will now be discussed in more detail with reference to the examples which are provided for exemplification only and which should not be considered limiting on the scope of the invention in any way.

### EXAMPLES

### Example 1: Visualisation of Fenestrations of LSEC Morphology

To study the morphology of in vitro LSECs a Scanning Electron Microscope (SEM) was utilised. LSEC fenestrations and sieve plates in primary LSECs cultured from young and old mice were resolved using SEM, sample images shown in the controls images in FIGS. 1 and 2.

Young and old C57B16 mice (n=3 young mice, age 3-4 and n=3 old mice, 20-24 months) were maintained under full SPF conditions and with ad libitum feeding. The study had the approval of the Sydney South West Area Health Service Animal Welfare Committee. Mice at 20-24 months are senescent. Animals were sacrificed withCO₂ and livers immediately perfusion-fixed via a 23G needle inserted into the portal vein. Liver tissue was fixed with1% glutaraldehyde/4% para-formaldehyde in PBS (0.1M sucrose).

Fenestrations ranged from 30-300 nm with an average diameter of 136 nm for young mice (3 months) and 124 nm for old mice (24 months).

Fenestrations were grouped into sieve plates (shown by * in FIG. 1) and contained 10-100 fenestrations in young mice and 5-50 in old mice. Young, compared to old mice, had an increased fenestration porosity and frequency, while old mice demonstrated greater expression of gaps (shown by # in FIG. 1) (>300 nm diameter holes).

### Example 2: Drug Treatments

Drug treatments and dosages (shown superimposed on their corresponding image in FIGS. 1, and 2) were performed by incubating liver cells at 37° C., 5% CO₂ for 30 mins using RPMI with or without dissolved drug. All images were taken by two blinded researchers using a SEM at 10,000×, scale bars of 1 μm are shown.

The control images show fenestrations grouped in sieve plates (*). Reduced fenestrations were observed between young and old mice. Gaps (#) (>300 nm) were present in old mice controls and promoted in simvastatin treatments. Bosentan, 2,5-Dimethoxy-4-iodoamphetamine (DOI), amlodipine and sildenafil treatments maintained sieve plates and increased fenestration density in both young and old mice. Cytochalasin D, Tumor necrosis factor-related apoptosis inducing ligand (TRAIL) and nicotinamide mononucleotide (NMN) treatments promoted increased fenestration density and maintained sieve plate fenestration clustering.

Old mice demonstrated greater fenestration sieve plate grouping following treatments with Cytochalasin D, TRAIL and NMN. 7-ketocholesterol (7KC) treatment promoted increased fenestrations but limited clearly definable sieve plates. Increased diameter of fenestrations was observed with 7KC and NMN treatments.

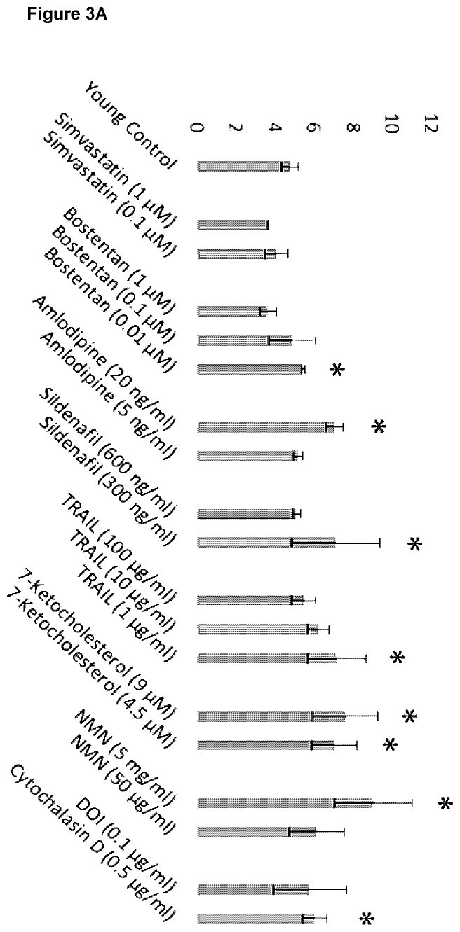

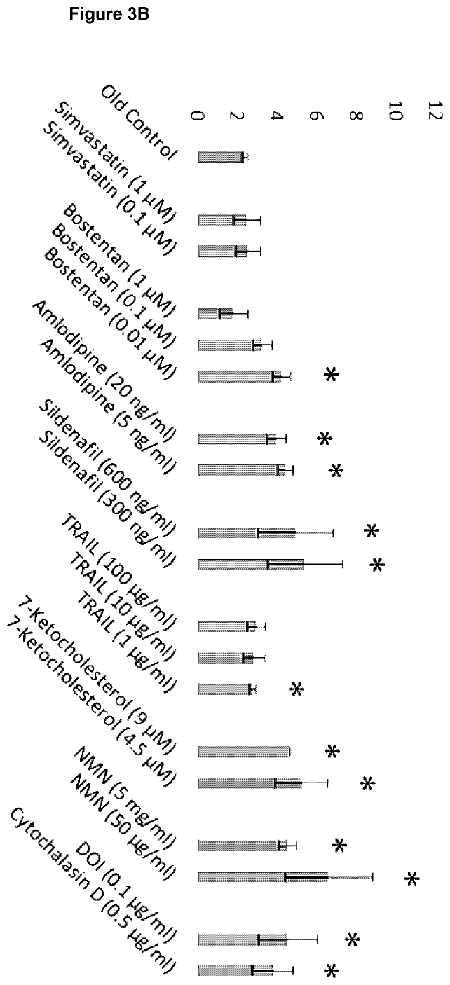

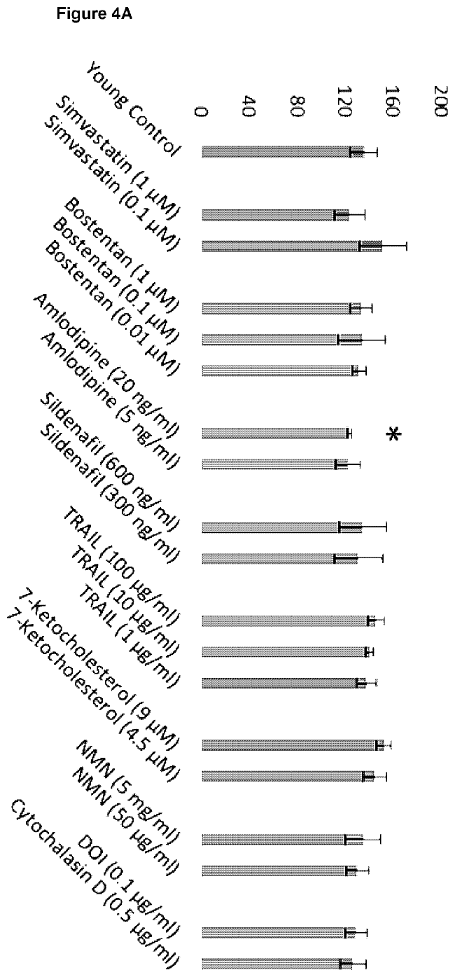

The effects of drug treatments on fenestration porosity, diameter and frequency are shown in FIGS. 3-5. From these Figures it can be seen that young control mice reported a fenestration porosity of 4.8±0.4%, (mean±SD) an average diameter of 135.9±11.1 nm and a frequency of 3.1±0.6 (number/100 μm2). Old mice had a significant reduction in porosity (2.4±0.1%; P<0.05) and frequency (1.8±0.3; P<0.05) compared to young mice, no significant differences were observed in diameter (Old: 124.4±6.2 nm; P=0.20).

FIGS. 3-5 shows the effects of Simvastatin, Bosentan, Amlodipine, Sildenafil, TRAIL, 7KC, NMN, DOI and Cytochalasin D on the porosity of LSEC fenestrations. Each data point represents the average±SD of 8 images (as shown in FIGS. 1 and 2), 616-3312 fenestration raw data points were collected per treatment. A fenestration <30 nm and gaps >300 nm were excluded from analysis. * shows P<0.05 using a paired t-test, n=2 for all groups except controls, Bosentan (1 μM), TRAIL groups and Cytochalasin D, these groups had n=3.

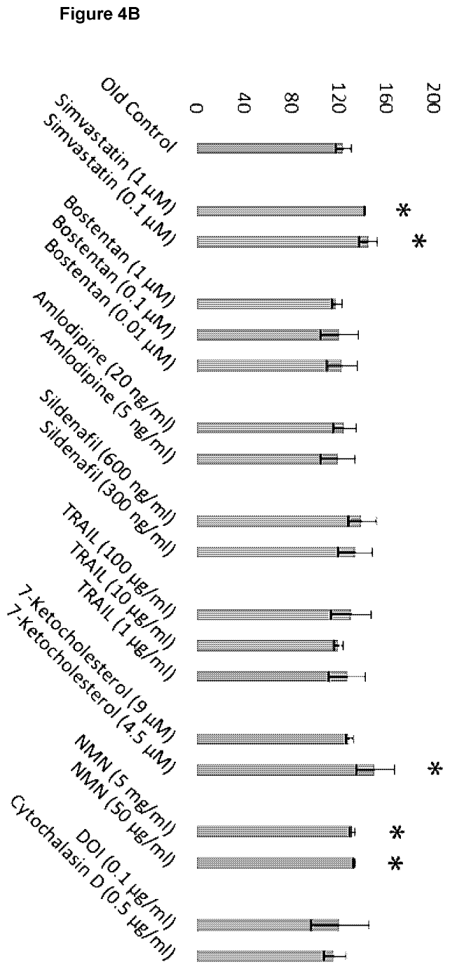

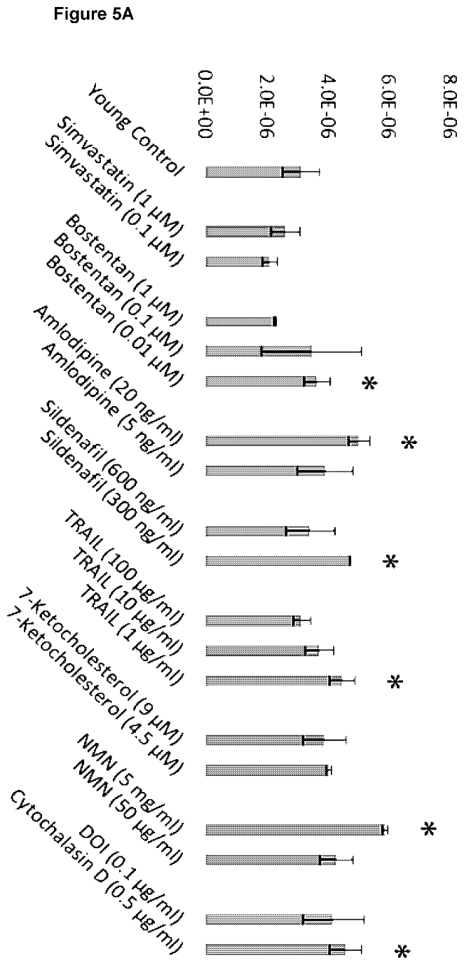

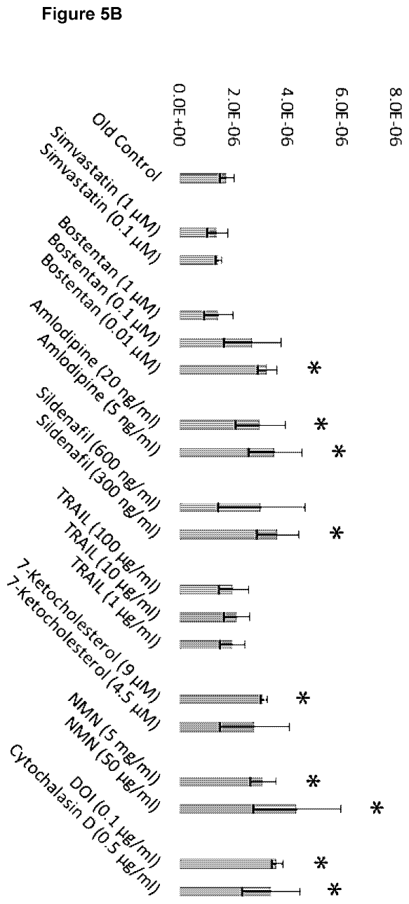

Cytochalasin D (0.5 μg/ml), DOI (0.1 μg/ml) and 7KC (9 μM) treatments show increased porosity in young (except DOI) and old mice (FIG. 3). Increased fenestration diameter was observed in 7KC (4.5 μM) treated LSECs in old mice only (FIG. 4). Fenestration frequency was increased in both young and old mice following Cytochalasin D treatment and in old mice only due to DOI and 7KC (9 μM) treatments (FIG. 5).

Nitric oxide (NO) pathway promotor drugs Bosentan (0.1 μM) and Sildenafil (300 ng/ml) promoted similar increased fenestration porosity in both young (5.4±0.1%; P<0.05, 7.1±2.2%; P<0.05) and old (4.2±0.4%; P<0.05, 5.4±1.9%; P<0.05) mice. Fenestration frequency showed a similar significant increase in both young and old mice. No changes in fenestration diameter were reported. Ca²⁺ inhibitor Amlodipine (20 ng/ml) increased fenestration porosity and frequency in young and old mice similarly to Bosentan and Sildenafil. Decreased fenestration diameter was also observed in young mice only (123.8±1.6 nm; P<0.05) (FIG. 4). Simvastatin, a NO pathway promoter via Kruppel-like factor 2, did not significantly change fenestration porosity or frequency, however an increased fenestration diameter (152.0±19.2 nm) was promoted with both Simvastatin treatments in old mice (FIG. 4).

Death receptor 4/5 promoter TRAIL (1 μg/ml) increased fenestration porosity and frequency in young (7.2±1.5%; P<0.05, 4.5±0.4; P<0.05) and porosity alone in old mice (2.7±0.1%; P<0.05). No changes in diameter were observed.

NMN increased fenestration porosity and frequency by the highest extent of the drug treatments examined. Dosages of 5 mg/ml in young mice and 50 μg/ml in old mice showed the greatest effects. In young mice, NMN treatment increased porosity to 9.1±2.0% (P<0.05) and frequency to 5.9±0.1 (P<0.05). In old mice, porosity increased to 6.6±2.2% (P<0.05) and frequency to 4.4±1.6 (P<0.05). Increased fenestration diameter significantly occurred in old mice (133.4±0.9 nm; P<0.05); this diameter was visually similar to that observed in young mice (FIG. 2).

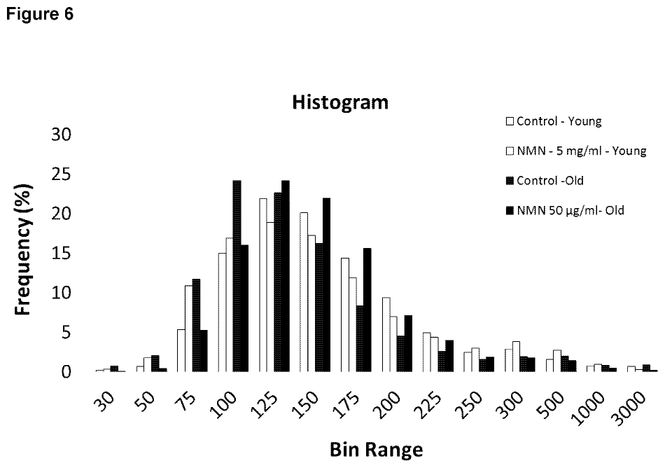

A histogram frequency of fenestration diameter was generated comparing control and NMN treatments in young and old mice (FIG. 6). In old mice, NMN treatment reported a peak frequency of 24% for a bin range of 101-125 nm. This result was similar to young control mice with a peak frequency of 22% in this range. Old control mice had a peak frequency of 24% for a bin range 76-100 nm.

### Example 3: Experimental Protocol for preparing Ag₂S Quantum Dots

Water soluble NIR-Ag₂S Quantum Dots (nanoparticles ˜5-10nm) were prepared for in vitro and in vivo studies.

Materials Used: Silver diethyldithiocarbamate Ag (DDTC), 1-Dodecanethiol, cyclohexane, α-Lipoic acid synthetic (thioctic acid), anhydrous ethanol, deionized water.

Equipment Used: Centrifugation machine, weighing machine, Corning Spin-X UF concentrators centrifugal filter, flat bottom flask, rubber septa, Magnetic heating plate, magnetic stir bar (mix the quantum dots dispersion), N₂ atmosphere, Sonicator.

The Ag₂S Quantum Dots were prepared according to the following protocol.

Step 1: Preparation of hydrophobic silver sulfide quantum dots were prepared as follows:
1. 0.02561 g of silver diethyldithiocarbamate (Soluble in pyridine P.T.) and 10 g of dodecanethiol (Soluble in water) were mixed in a flask at room temperature.
2. Oxygen was removed with vigorous magnetic stirring under vacuum for 5 min.
3. The solution should was heated to 200° C. at a heating rate 15° C./min and kept at 200° C. for 1 h under N₂ atmosphere.
4. The solution was allowed to cool to room temperature naturally. Subsequently 50 ml of ethanol was poured into the solution.
5. Then resultant mixture was centrifuged at 6729 g for 20min and the pellet was washed and dispersed in cyclohexane.

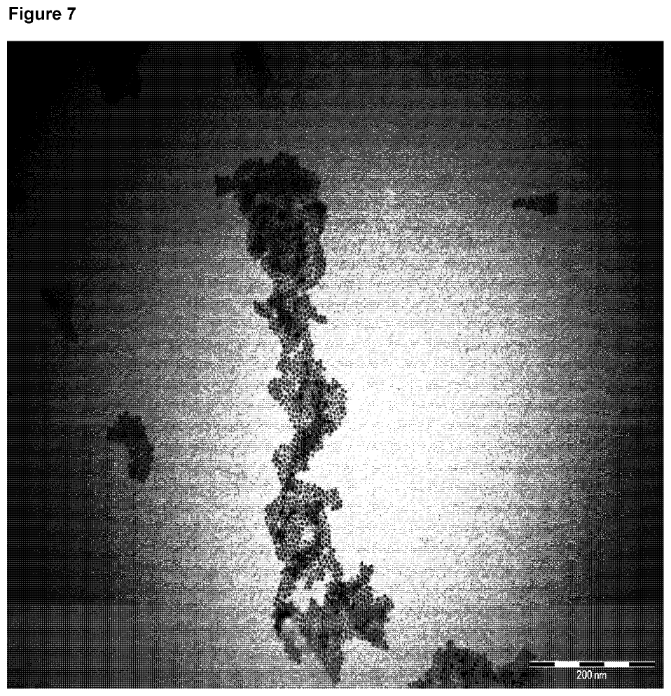

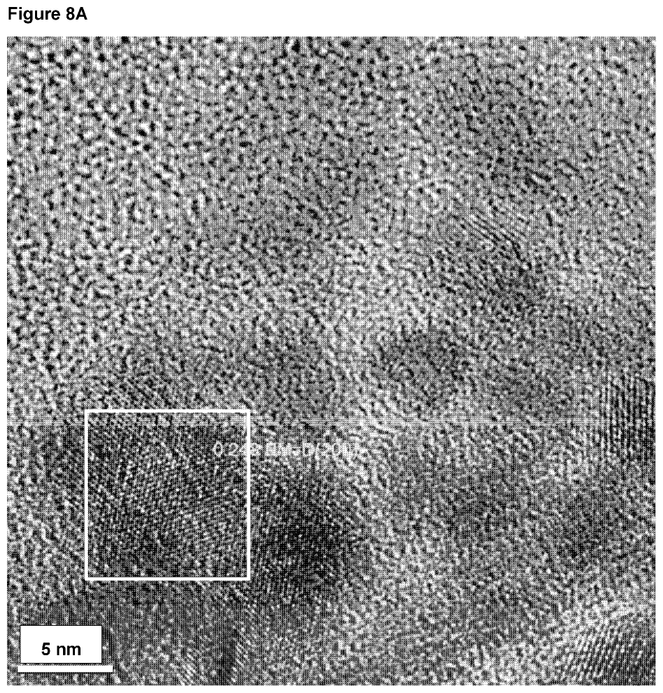

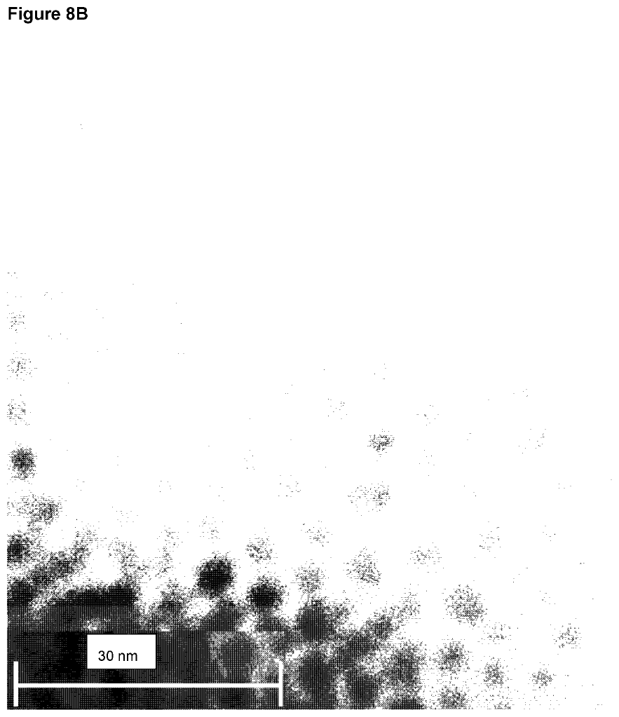

The cyclohexane dispersion contains monoclinic Ag₂S quantum dots which can be identified by using X-ray diffraction and Transmission electron microscopy (TEM). See FIGS. 7 and 8.

Step 2: Preparing hydrophilic silver sulfide quantum dots
1. 0.15 g of thioctic acid and 15 ml of ethanol were added to 0.05 mmol of the cyclohexane dispersion from step 1 and the resultant mixture sonicated in an ultrasonic cleaner for 4 h. (Thioctic acid is soluble in ethanol).
2. The sonicated mixture was centrifuged at 2691 g for 20 min, washed with deionized water and redispersed in deionized water. The sample contains water soluble Ag₂S particles (quantum dots) approximately 5-10 nm in diameter. The particles have strong fluorescence emission at 1100-1200nm with an incident wavelength of 785nm.

### Example 4: Conjugation of Quantum Dots with a Therapeutic

Quantum were conjugated to a therapeutic (cytochalsin D) according to the following protocol:
1. 0.1 mg of the Ag₂S quantum dots (QD) from Example 1 was dispersed in in 200 μL of dimethyl sulfoxide (DMSO).
2. 1.15 mg (0.01 mmol) of sulfo-N-hydroxysuccinamide (NHS) dissolved in 50 μL of DMSO was added to the mixture from step 1 and mixed by stirring.
3. 1.91 mg (0.01 mmol) sample of 1-ethyl-3-(3-dimethylaminopropyl)carbodiimide (EDC) was dissolved in 50 μL of DMSO and added into the QD-NHS/DMSO solution from step 2.
4. The mixture from step 3 was kept for 1 h in the dark with stirring.
5. The surface activated Ag₂S QDs produced in step 4 were centrifuged and washed with DMSO twice and further dispersed in DMSO.
6. 2×10⁻⁹ mol of Cytochalasin D protein in PBS buffer was conjugated with the EDC/NHS-activated Ag₂S QDs from step 5.

### Example 5: Ag₂S QDs Label LSECs

Isolated LSEC were seeded in 96-well plates (1×10⁴ cells per well) and subsequently incubated for 24 h 37° C. The cells were incubated with 25 micrograms of Ag₂S QDs from Example 1 at 37° C. for 15 minutes or 24 h.

After incubation the cells were washed three times with PBS (pH 7.0) to remove unbound QDs and then prepared for electron microscopy. Cells were fixed using 2.5% glutaraldehyde in 0.1M cacodylate buffer for 2 hours at room temperature, washed with 0.1M cacodylate buffer and postfixed in osmium tetroxide for 1 hour. Water was removed for the cells using increasing concentrations of ethanol with final substitution into Spurr's resin for embedding. 70-nm ultrathin sections were cut using an ultramicrotome, sections. Sections were examined with a FEI/Philips CM-200 electron microscope for detection of the presence of QDs

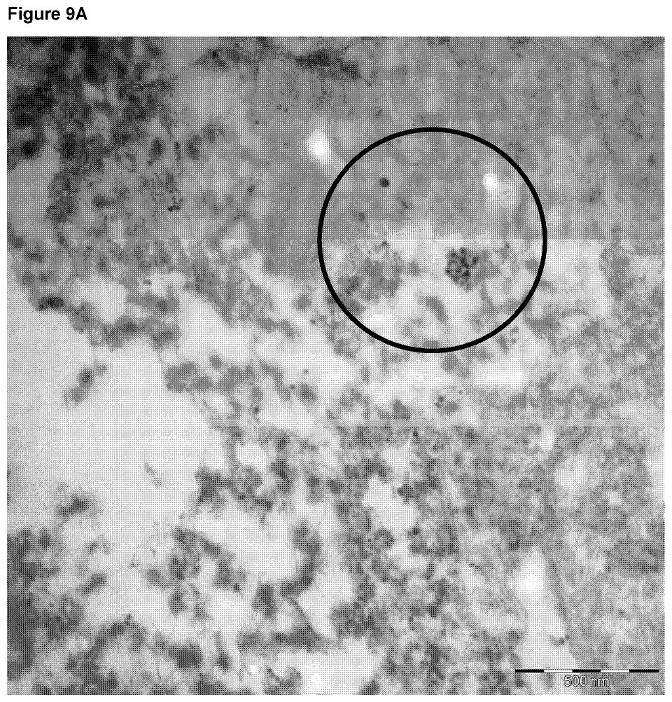

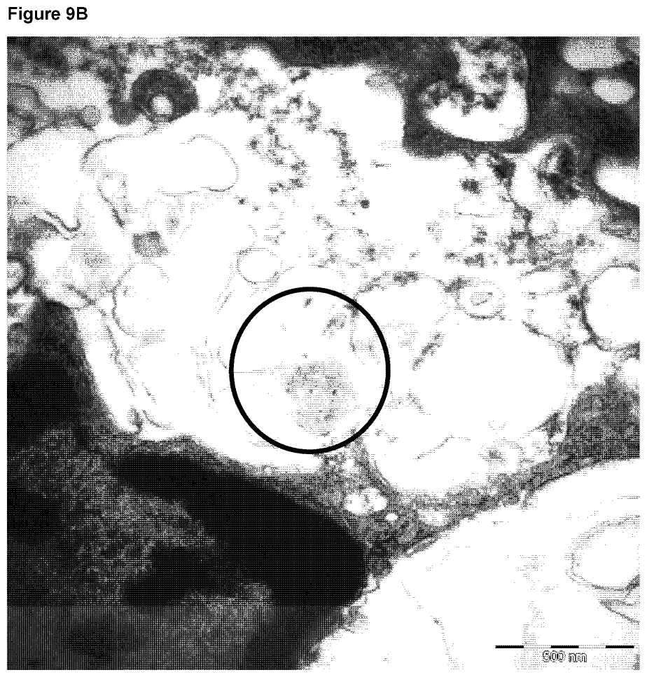

Electron micrographs of LSECs labelled with Quantum dots are shown in FIG. 9.

### Example 6: Ag₂S QDs Label Cells in the Intact Liver

After anesthesia, livers of mice are perfused via the portal vein using Krebs Henseleit bicarbonate buffer (1% albumin, 10 mM glucose, pH 7.4) containing 250 micrograms of QDs

After 5 minutes of perfusion with QDs, livers are perfused with fixative and the livers analysed for QDs distribution using transmission electron microscopy.

The livers were perfusion-fixed with 3% glutaraldehyde and 2% paraformaldehyde in 0.1 M sodium cacodylate buffer and were then processed and embedded in Spurrs Resin prior to ultrathin sectioning and examination using the FEI/Philips CM-200 electron microscope.

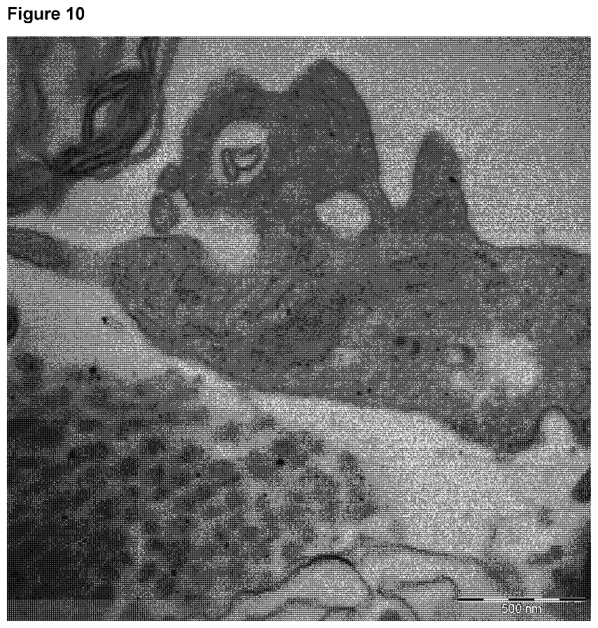

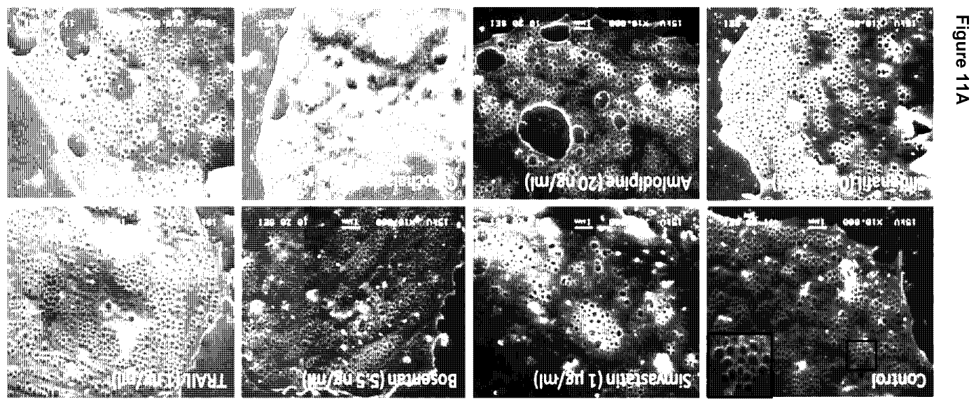

Electron micrographs of liver sections labelled with Quantum dots are shown in FIG. 10.

### Example 7: Quantum Dot (QD) Preparation Paper Draft Methods Ag₂S QD Synthesis

As described above Ag₂S QDs were synthesised with 0.1-0.3 grams silver diethyldithiocarbamate with 12 ml 1-dodecanethiol mixed under vigorous magnetic stirring. An N₂ vacuum was created to remove oxygen from the mixture, followed by an Ar vacuum to remove N₂. Ag₂S QD solution was heated to 180-210° C. at a rate of 10-15° C. per min and held at this temperature for 1-60 mins. Following synthesis 50-100 ml of EtOH was added to the solution with Ag₂S QDs centrifuged at 4000-28000 RPM for 30 mins.

### Ag₂S QDs Washing

Ag₂S QDs were resuspended in cyclohexane and washed twice with acetone and twice with EtOH. Each wash resulted in precipitation of Ag₂S QDs at 4000 RPM. Alternatively, separation can be obtained by mixing equal volume of MQ to EtOH or acetone resulting in a two layer non-miscible solution with Ag₂S QDs in the cyclohexane layer.

### Radiolabelling of Ag₂S QDs

Ag₂S QDs were synthesised, washed dispersed in cyclohexane as described above. QDs (50 mg) were incubated at room temperature with 5 pCi 3H Oleic Acid for 48 hrs under Ar gas with vigorous stirring. Following incubation QDs were washed with 3 times with acetone to precipitation of the QDs, centrifuged at 3000 RPM for 5 mins and redispersed in cyclohexane.

### Aqueous Phase Transfer

Radiolabelled QDs in cyclohexane were mixed 1:1 (v/v) with acetone under magnetic stirring. 1 ml of 3-MPA was added per 50 mg of Ag₂S QDs. Ag₂S QDs were incubated at room temperature for 1 hr, mixed with 50 ml ethanol and centrifuged at 3000 RPM for 5 mins. The pellet was washed with 70% ethanol in water 3 times and dispersed in MQ. Following phase transfer QDs were diluted to 10 mM solutions for storage at 4° C. in the dark.

### FSA Coating

10 mM Ag₂S QDs were mixed with 10 mM EDC and 10 mM NHS in a reaction vial under heavy mixing for 1 hr. Following this the pH was increased to 9 and 10 mM fibroblast surface antigen (FSA), FSA-488 or FSA-647 was added to the solution. The mixture for incubated at room temperature for 4 hrs. The mixture was transferred to snakeskin dialysis tubing 3500-10000 molecular weight filters and dialysed with PBS for 2-3, 5-6 and overnight at 4° C. in the dark. Following dialysis solutions were collected from the tubing and storied at 4° C. until use.

The Ag₂S QDs have the following characteristics:

TABLE 1
QD characteristics

| Quantum Dot | 1 | 2 | 3 |
|---|---|---|---|
| Base material | Ag₂S | Ag₂S | Ag₂S |
| Size | 4.04 ± 1.56 | 6.0 ± 1.67 | 30.0 ± 1.34 |
| Zeta | −25.8 ± 0.8 | −31.2 ± 1.3 | −28.5 ± 0.5 |
| Coating | FSA-488 | FSA-488 | FSA-488 |
| | fluorophore | fluorophore | fluorophore |

### Mice Gavage

3-4 month old male C57/B16 mice were obtained from the Animal Resource Centre in Perth, Western Australia. Animals were housed at the ANZAC Research Institute animal house on a 12 hour light/dark cycle and provided with ad libitum access to food and water. Mice were not fasted prior to gavage with 100 ml 10 mM 3H-Ag₂S-FSA-488 QDs. Blood was collected at 0, 10, 20 and 30 mins post gavage with mice euthanized by a single intraperitoneal injection with 100 mg/kg ketamine and 10 mg/kg xylazine in saline at 30-60 mins post gavage. 200-250 mg of tissue was collected from the liver, spleen, kidney, lung and small bowel. Tissue samples were weighted and mixed in a reaction vial with 1 ml Solvable solution and incubated at 60° C. for 2 hrs to dissolve the tissue. 0.2 ml 30% H₂O₂ were added to samples to reduce the dark colour saturation. Samples were mixed with 10 ml scintillation fluid.

### LSEC Isolation

Mouse hepatocyte, LSEC, HSC and Kupffer cells isolation was performed by perfusion of the liver with collagenase. Hepatocytes were removed by three 10 min centrifugations at 50×g Non-parenchymal and dead cells were removed from the hepatocyte and LSEC fractions by separate two-step Percoll gradients with Küpffer cells removed from the LSEC fraction by selective adherence to plastic. Cells were suspended in PBS followed by cell counting, centrifuged and weighted, following either (i) mixing in a reaction vial with 1 ml solvable solution and prepared as stated above for radiolabelled detection or (ii) unaltered for analysis in flow cytometry (samples for flow cytometry were not radiolabelled).

### Flow Cytometry

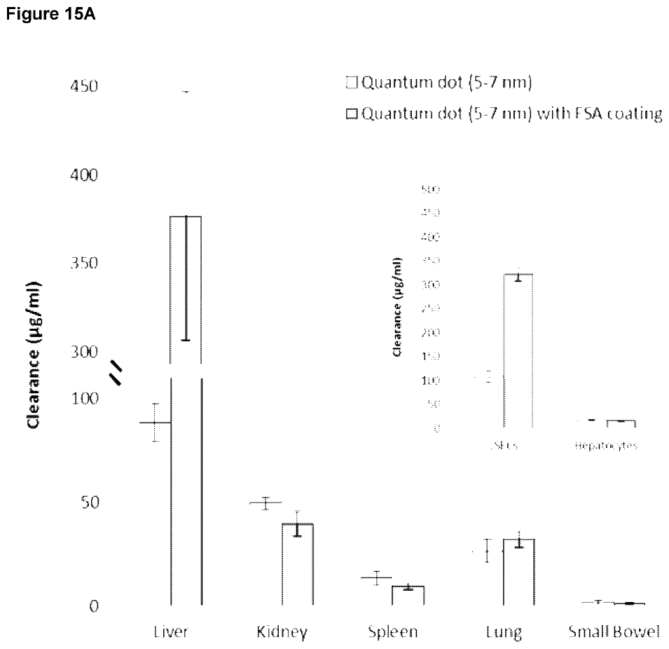

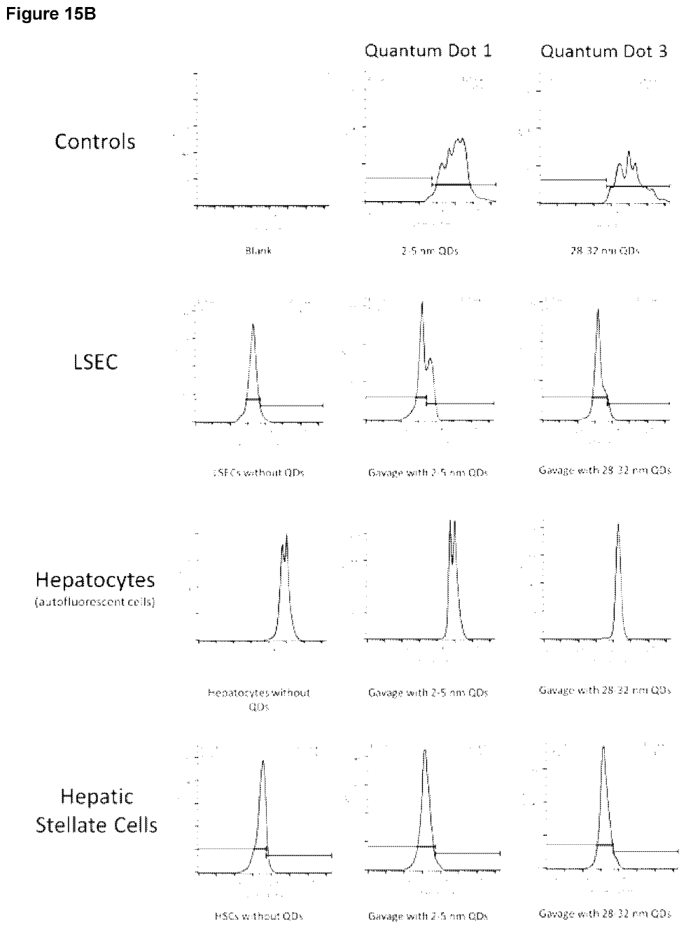

Flow cytometry was performed on an BD-Accuri flow cytometer (BD biosciences, Australia) with data analysed on FlowJo (v10, FlowJo LLC, ON, USA). Samples were diluted to 1.0×10⁶ cells/ml with an additional 2 serial half dilutions. Size execution criteria were applied in addition to the isolation preparation as described above. 100,000 events were collected per dilution with events limited to size criteria. The data in FIG. 15A shows the sample data following size execution.

### 3H-Radiolabelled Activity Analysis

Radioactivity was measured using a scintillation counter (Tricarb 2100 TR) (5 mins per sample, 5-10 ml scintillation fluid). The data in FIG. 15B shows the level of radioactivity present in blood relative to radiolabelled Ag₂S QDs alone. Ag₂S QDs clearance was determined by the expression of radioactivity between organs and the blood sample per ug/ml based on the tissue weight. All samples were run in triplicate.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### Example 7: Effect of Agents on Fenestrations in Isolated LSECs from Young and Old Mice

This Example was performed to investigate the action of several agents on fenestrations in isolated LSECs from young (3-4 month) and old (18-24 month) mice, in order to: (1) describe the different mechanisms that regulate fenestrations and; (2) identify drugs that reduce age-related loss of fenestrations. We studied drugs that act on the pathways that influence NO (sildenafil, amlodipine, simvastatin, serotonergic pathway/phospholipase C (DOI), endothelin receptor (bosentan), death receptor (TRAIL) and NAD+ (nicotinamide mononucleotide NMN)) in mice using scanning electron microscopy (SEM) and dSTORM. Established fenestration-active agents that act on the actin cytoskeleton and lipid rafts (cytochalasin D and 7-ketocholesterol respectively) were employed as positive controls. The results indicate that by targeting the NO pathway and inducing actin remodelling re-fenestration was promoted in old mice. Agents that ameliorate age-related defenestration may have therapeutic potential for age-related dyslipidaemia and insulin resistance.

### Materials and Methods

3-4 and 18-24 month old male C57/B16 mice were obtained from the Animal Resource Centre in Perth, Western Australia. Animals were housed at the ANZAC Research Institute animal house on a 12 hour light/dark cycle and provided with ad libitum access to food and water. Mice were not fasted prior to euthanasia by a single intraperitoneal injection with 100 mg/kg ketamine and 10 mg/kg xylazine in saline. The study was approved by the Animal Welfare Committee of the Sydney Local Health District and was performed in accordance with the Australian Code of Practice for the care and use of animals for scientific research (AWC 2016/009). All information provided accords with the ARRIVE guidelines.

Reagents included: collagenase (Type 1, cat no: 47D17410A, ProSciTech, AUS), RPMI-1640 (Sigma-Aldrich, AUS), percoll (Sigma-Aldrich, AUS), cytochalasin D (cat no: c8273, Sigma-Aldrich, AUS), TRAIL (cat no: 375-TL-010, R&D systems, AUS), bosentan (cat no: S4220, Selleckchem, Tex., USA), 7-ketocholesterol (cat no: c2394, Sigma-Aldrich, AUS), 2, 5-dihydroxl-4-isoamphetamine (cat no: 13885, Cayman Chemicals, AUS), simvastatin (cat no: S6196, Sigma-Aldrich, AUS), sildenafil citrate (cat no: PZ0003, Sigma-Aldrich, AUS), nicotinamide mononucleotide (gift from Dr Lindsay Wu, UNSW, AUS), amlodipine besylate (cat no: A5605, Sigma-Aldrich, AUS) and VEGF (cat no: V4512, Sigma-Aldrich, AUS). Stains included Alexa Fluor 488 phalloidin (cat no: A12379 Thermo Fisher, AUS), phosphorylated-eNOS (cat no: 9571, Cell signalling Technology, AUS), eNOS (cat no: 610296, BD Biosciences, AUS) Alexa Fluor 488 Goat anti-Rabbit, Cy3 Goat anti-Mouse (cat no: R-37116, A-11003; Thermo Fisher, AUS). Assays were performed using In Vitro Toxicology Assay Kit, MTT based (cat no: TOX1-1KT, Sigma-Aldrich, AUS) and Cyclic GMP ELISA kit (cat no: 581021, Cayman Chemicals, AUS).

As described previously (Cogger et al. J e52698, 2015 https://dx.doi.org/10.3791/52698), mouse LSEC isolation was performed by perfusion of the liver with collagenase. Non-parenchymal cells were removed by a two-step Percoll gradient and Kupffer cells were removed by selective adherence to plastic. LSECs (seeded at 0.5×106 cells/cm2) were cultured (37° C., 5% CO₂) in serum free RPMI-1640 for 3.5 hours before use.

Cells were treated with various agents for 30 minutes to determine effects on fenestrations. All agents were dissolved in serum-free RPMI media. All experiments were performed in triplicate for both young and old mice. Actin was disrupted with 0.5 μg/ml cytochalasin D and lipid rafts were disrupted with 3.6 and 1.8 μg/ml 7-keocholesterol; dosages were selected. The NO pathway was promoted with sildenafil (0.6, 0.3, 0.15, 0.05 and 0.015 μg/ml), amlodipine (40, 20, 10, 5 and 1 ng/ml), and simvastatin (1 and 0.1 μg/ml). The serotonergic/phospholipase C pathway was promoted with DOI (0.1 μg/ml) and endothelin receptors were inhibited by bosentan (550, 55 and 5.5 ng/ml) Death receptor 4 was promoted with TRAIL (100, 10. 1, 0.1 and 0.01 ng/ml) and NAD+ was promoted with NMN (5000, 50, 10, 1 and 0.1 μg/ml).

SEM was performed as previous described (Corbin et al J Biol Chem 274: 13729-13732, 1999.). LSECs were fixed in 2.5% glutaraldehyde in 0.1M sodium cacodylate buffer, osmicated, dehydrated in graded ethanol and hexamethyl disilazane, mounted on stubs, sputter coated with platinum and examined using a JEOL 6380 Scanning Electron Microscope (JEOL Ltd, Japan). Images at 10,000× magnification were collected by a blinded observer and used to measure fenestration diameter and LSEC porosity using Image J (NIH, MD, USA). Between 616-3312 fenestrations were counted per treatment. Fenestrations less than 30 nm and gaps more than 300 nm were excluded from analysis. Porosity was defined as the percentage of the cell membrane covered with fenestrations. Frequency was defined at the number of fenestrations per 1 pmt.

dSTORM imaging was performed using an in-house microscope. LSECs were prepared for dSTORM by washing twice with PBS and fixation with 4% paraformaldehyde for 30 mins. Then LSECs were washed twice with PBS, permeabilised with Triton-X for 90 secs, blocked with 5% bovine serum albumin for 1 hour, and stained with Alexa Flour phalloidin 488 (1:40) for 20 min prior to imaging. Cells were washed using PBS with 0.1% Tween and placed in OxEA buffer (30) for dSTORM visualisation and image capture. The dSTORM used 488 and 647 nm excitation from diode-pumped lasers (Coherent Inc, CA, USA). Excitation was delivered via a 1.49NA 60× oil-immersion TIRF objective (Olympus Australia, AUS). Fluorescence was captured on two separate sCMOS cameras (Imaging Development Systems GmbH, Germany). Data was collected for up to 40,000 images at around 75 fps. 5-8 whole cell images were collected by a blinded observer for each treatment dosage and processed using rapidSTORM open source software (Wolter et al. Nat Methods 9: 1040, 2012.). Each image was examined for all sieve plates and actin structures. Densitometry measurements were performed using 5-8 dSTORM images with data analysis performed using Image J software (NIH, MD, USA).

Immunofluorescence was performed on LSECs fixed with 4% paraformaldehyde. LSECs were permeabilised with Triton-X for 90 secs, blocked with 5% normal goat serum for 1 hour and incubated with (1:100) phosphorylated-eNOS and (1:100) eNOS overnight at 4° C. LSECs were washed twice with PBS and incubated with Alexa Fluor anti-rabbit 488 and Alexa Fluor anti-mouse Cy3 secondary antibodies. Cells were washed with PBS and mounted using Vector Mount with DAPI. Slides were examined at 63× magnification using a Leica SP8 inverted scanning confocal microscope with Type F immersion oil (cat: 11513859) and images captured using LAS software (Leica Microsystems CMS GmbH, Germany) by a blinded observer. Images were analysed using ImageJ (NIH, MD, USA).

Assays for MTT and cGMP were performed as instructed by the kit. Briefly, MTT assays were performed following drug treatments. Cells were washed with PBS and incubated with RPMI media containing 100 pg MTT solution. Cells were incubated at 37° C. for 4 hrs and lysed with 200 pl solubilisation solution, 30 mins colour development followed and measured at 570 nm using a spectrophotometer. cGMP assays were also performed after drug treatments. Cells were washed with PBS and lysed with 0.1M HCl. Following sample collection the sample was acetylated and prepared with kit reagents. Samples were incubated for 18 hrs at 4° C. before examination at 410 nm with a spectrophotometer.

Statistical analysis between drug treatments experiments and actin/NOS densitometry was performed comparing multiple groups using Kruskal-Wallis tests with a post hoc Dunn's method (SPSS v21, IBM Analytics, AUS) with P<0.05 considered significant; P<0.1 are also highlighted in the results. Non-parametric statistics were used due to the number of mice used in this study with analysis of previous data demonstrating this sample size produces a statistical power of 80-95% to discriminate between interventions. Individual specifications of analyses are described in figures legends. All data are presented as mean±SD. Experimental design and analysis were performed in accordance with the APS guidelines described in Curran-Everett and Benos DJ. Adv Physiol Educ 31: 295-298, 2007.

### Results

### Young and Old Controls

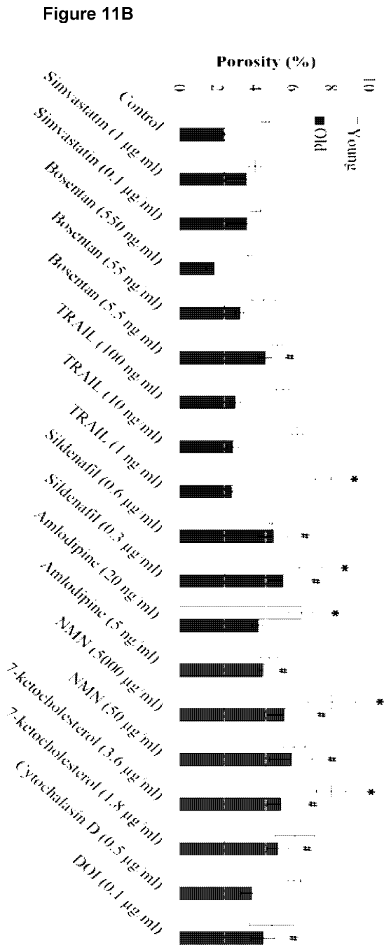

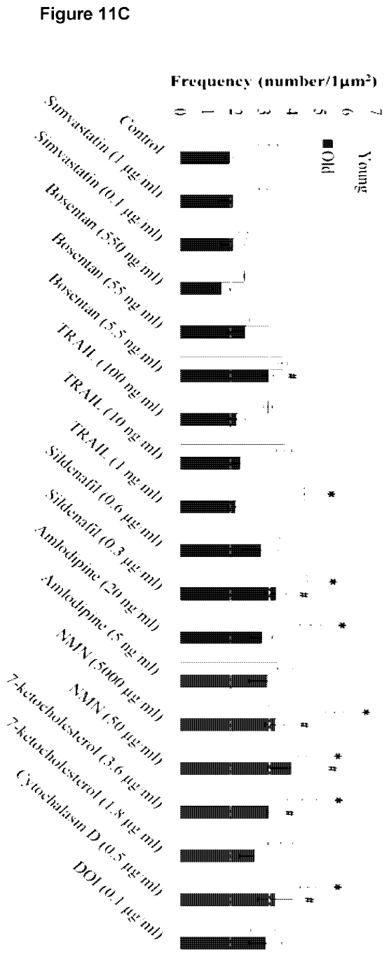

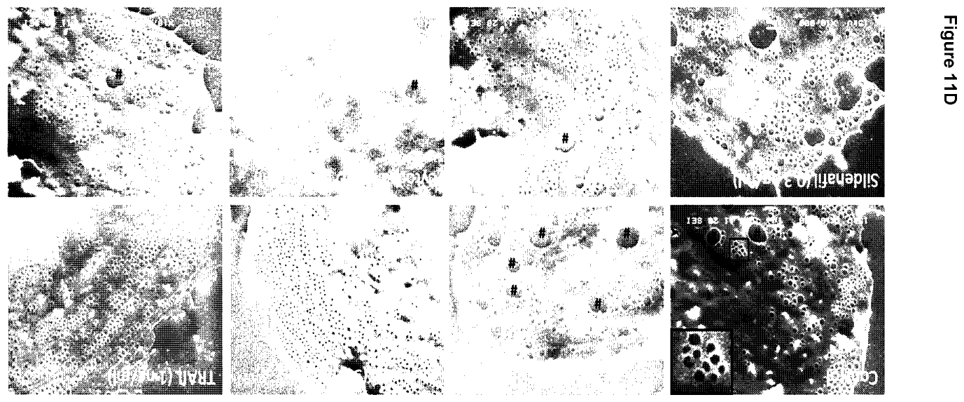

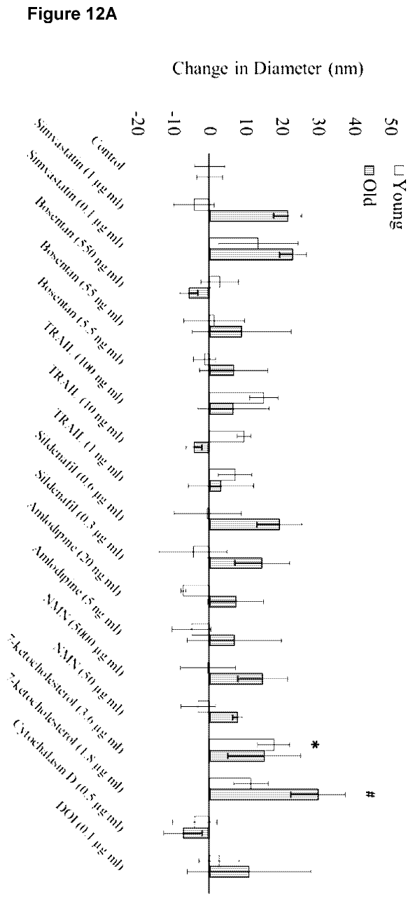

SEM of isolated LSECs from young and old mice confirmed the technical success of LSEC preparations as shown in FIG. 11A. As expected, LSECs from old mice had reduced porosity when compared to young LSECs (Porosity: young 4.6±0.3%, vs old 2.4±0.1%; P=0.023, N=3 per group, FIG. 11B), with a greater number of gaps (>300 nm diameter, indicated by # in FIG. 11D). There was no significant difference in fenestration diameter with age (young 130.9±7.2 nm vs old: 124.4±6.2 nm; P=0.20, FIG. 2). There was a reduction in fenestration frequency with age (young: 3.1±0.6 fenestrations per 1 μm2 vs old 1.8±0.3; P=0.033, FIG. 11C). This indicates that age-related defenestration in these mice was largely secondary to reduced frequency of fenestrations rather than a reduction in diameter. In FIG. 11 dotted lines show young and old control levels. Drug treatments: simvastatin, bosentan, TRAIL, sildenafil, amlodipine, NMN, 7-ketocholesterol, cytochalasin D and DOI. All treatments were incubated at 37° C., 5% CO₂ for 30 mins using RPMI with or without dissolved drug. SEM images were taken by two blinded researchers at 10,000× (sample images shown in panel A, D) and used to manual count fenestration porosity and frequency. Each data point represents the average±SD of 8 images, using 616-3312 fenestration raw data points per treatment. All fenestrations <30 nm and gaps >300 nm were excluded from analysis. * Shows P<0.05 compared to young control; # shows P<0.05 compared to old control. Statistics were performed using Kruskal-Wallis with post-hoc Dunn's test to compare between groups, n=3 for all groups. (D) Sample SEM images of drug treatments in old mice. Scale bars of 1 μm are shown. Gaps (#) (>300 nm) were present in control and increased in simvastatin 1 μM treatments.

### Effects of Agents on Fenestrations

Treatment with sildenafil, NMN and 7-ketocholesterol led to significant increases in porosity and fenestration frequency in both young and old LSECs (FIG. 11B-C, Table 2, 3). Cytochalasin D significantly increased frequency but not porosity in young and old LSECs (FIG. 11B-C, Table 2, 3). LSECs from old mice only were responsive to bosentan and DOI. LSECs from young mice only demonstrated significant increases in porosity and frequency following TRAIL and amlodipine treatments. Overall greater fold changes in porosity and frequency were observed in LSECs from old mice compared to young mice. The greatest changes on old mice were promoted by NMN 50 pg/ml treatment, porosity increased by 2.5-fold and frequency by 2.25-fold (FIG. 11B-D)

TABLE 2
Young mice data: **shows P < 0.01, *shows P < 0.05, ^{#}shows P < 0.1;
using Kruskal-Wallis with post-hoc Dunn's test to compare
between groups. All data shown as mean ± SD

| Drug | Porosity | Diameter | Frequency |
|---|---|---|---|
| Treatment | (%) | (nm) | (no/area) |
| Control | 4.55 ± 0.34 | 130.89 ± 7.23 | 3.15 ± 0.60 |
| Amlodipine | 6.44 ± 1.14* | 123.66 ± 1.18 | 4.67 ± 0.65* |
| (20 ng/ml) | | | |
| Amlodipine | 4.73 ± 0.80 | 125.87 ± 9.30 | 3.47 ± 1.00 |
| (5 ng/ml) | | | |
| Bosentan | 3.66 ± 0.41 | 133.73 ± 9.03 | 2.30 ± 0.04 |
| (550 ng/ml) | | | |
| Bosentan | 4.48 ± 1.12 | 132.12 ± 9.03 | 3.20 ± 1.23 |
| (55 ng/ml) | | | |
| Bosentan | 5.18 ± 0.45 | 129.55 ± 5.46 | 3.66 ± 0.30 |
| (5.5 ng/ml) | | | |
| Cytochalasin D | 6.05 ± 0.62^{#} | 126.78 ± 10.68 | 4.57 ± 0.51* |
| (0.5 μg/ml) | | | |
| DOI (1 μg/ml) | 1.45 ± 1.27 | 135.64 ± 21.21 | 0.83 ± 0.75 |
| DOI (0.1 μg/ml) | 4.91 ± 2.02 | 133.57 ± 9.78 | 3.40 ± 1.56 |
| NMN (5000 μg/ml) | 8.05 ± 2.23* | 130.48 ± 13.21 | 5.53 ± 0.58* |
| NMN (50 μg/ml) | 6.08 ± 1.00 | 127.68 ± 8.19 | 4.53 ± 0.58* |
| Sildenafil | 4.92 ± 0.35 | 130.33 ± 16.02 | 3.56 ± 0.61 |
| (0.6 μg/ml) | | | |
| Sildenafil | 6.34 ± 2.09* | 126.34 ± 16.36 | 4.54 ± 0.34* |
| (0.3 μg/ml) | | | |
| Simvastatin | 3.99 ± 0.55 | 126.55 ± 9.64 | 2.83 ± 0.51 |
| (1 μg/ml) | | | |
| Simvastatin | 4.05 ± 0.41 | 144.35 ± 18.95 | 2.42 ± 0.55 |
| (0.1 μg/ml) | | | |
| TRAIL (100 ng/ml) | 5.49 ± 0.63 | 145.87 ± 6.77 | 3.15 ± 0.27 |
| TRAIL (10 ng/ml) | 6.24 ± 0.52^{#} | 140.36 ± 3.22 | 3.73 ± 0.46 |
| TRAIL (1 ng/ml) | 7.17 ± 1.49* | 137.95 ± 8.12 | 4.47 ± 0.43* |
| 7-ketocholesterol | 8.03 ± 1.37* | 148.59 ± 7.70* | 4.36 ± 0.93* |
| (3.6 μg/ml) | | | |
| 7-ketocholesterol | 6.10 ± 1.82 | 142.35 ± 8.02 | 3.59 ± 0.78 |
| (1.8 μg/ml) | | | |

TABLE 3
Old mice data: **shows P < 0.01, *shows P < 0.05, ^{#}shows P < 0.1;
using Kruskal-Wallis with post-hoc Dunn's test to compare
between groups. All data shown as mean ± SD

| Drug | Porosity | Diameter | Frequency |
|---|---|---|---|
| Treatment | (%) | (nm) | (no/area) |
| Control | 2.40 ± 0.14 | 124.35 ± 6.15 | 1.77 ± 0.25 |
| Amlodipine | 3.98 ± 0.48* | 125.00 ± 13.36 | 3.00 ± 0.66 |
| (20 ng/ml) | | | |
| Amlodipine | 4.44 ± 0.29* | 119.67 ± 22.63 | 3.56 ± 1.07* |
| (5 ng/ml) | | | |
| Bosentan | 1.86 ± 0.72 | 118.64 ± 4.36 | 1.46 ± 0.54 |
| (550 ng/ml) | | | |
| Bosentan | 3.21 ± 0.36 | 121.14 ± 23.80 | 2.31 ± 1.03 |
| (55 ng/ml) | | | |
| Bosentan | 4.53 ± 0.59* | 131.03 ± 16.29 | 3.14 ± 0.35* |
| (5.5 ng/ml) | | | |
| Cytochalasin D | 3.82 ± 1.01 | 117.04 ± 9.26 | 3.39 ± 1.07* |
| (0.5 μg/ml) | | | |
| DOI (1 μg/ml) | 1.31 ± 0.47 | 155.28 ± 15.33* | 0.67 ± 0.31 |
| DOI (0.1 μg/ml) | 4.44 ± 1.07* | 135.27 ± 29.71 | 3.06 ± 1.00* |
| NMN (5000 μg/ml) | 5.55 ± 1.75* | 139.05 ± 11.97 | 3.39 ± 0.60* |
| NMN (50 μg/ml) | 5.92 ± 1.94* | 132.12 ± 2.28 | 3.95 ± 1.35* |
| Sildenafil | 4.97 ± 1.34* | 143.69 ± 10.80 | 2.88 ± 01.16 |
| (0.6 μg/ml) | | | |
| Sildenafil | 5.49 ± 1.33* | 138.91 ± 13.05 | 3.41 ± 0.68* |
| (0.3 μg/ml) | | | |
| Simvastatin | 3.54 ± 1.86 | 145.96 ± 6.55* | 1.88 ± 0.86 |
| (1 μg/ml) | | | |
| Simvastatin | 3.56 ± 1.75 | 147.47 ± 6.47* | 1.89 ± 0.74 |
| (0.1 μg/ml) | | | |
| TRAIL (100 ng/ml) | 2.97 ± 0.46 | 130.88 ± 17.00 | 2.03 ± 0.53 |
| TRAIL (10 ng/ml) | 2.85 ± 0.53 | 120.08 ± 3.74 | 2.13 ± 0.48 |
| TRAIL (1 ng/ml) | 2.79 ± 0.14 | 127.50 ± 15.53 | 1.97 ± 0.49 |
| 7-ketocholesterol | 5.34 ± 1.17* | 139.46 ± 17.45 | 3.15 ± 0.08* |
| (3.6 μg/ml) | | | |
| 7-ketocholesterol | 5.19 ± 0.95* | 154.41 ± 13.07* | 2.65 ± 0.94 |
| (1.8 μg/ml) | | | |

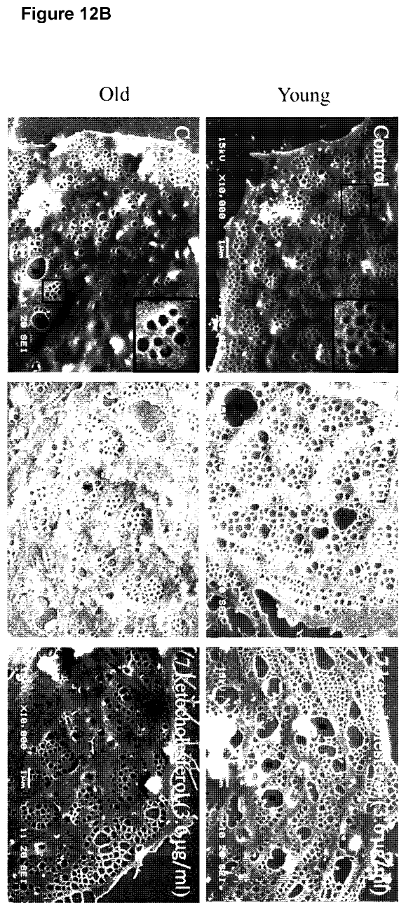

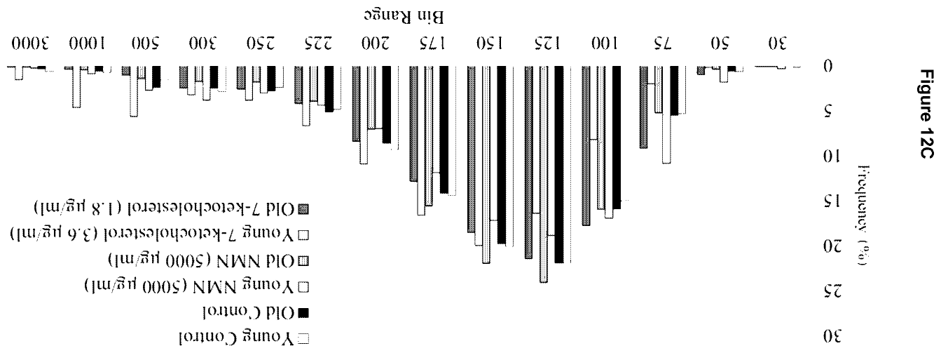

There were significant differences in the responses of LSECs to different drug agents and dosages. In young mice, sildenafil (0.3 μg/ml), amlodipine (20 ng/ml) and TRAIL (1 ng/ml) demonstrated increased fenestration numbers and overall fenestrated cell area with some disruption to sieve plate formation (FIG. 1); higher dosages of sildenafil and TRAIL did not promote greater changes in fenestration porosity or frequency. Gap formation was apparent following treatment with amlodipine, 7-ketocholesterol and NMN (indicated by # in FIG. 11A and FIG. 12B). Following NMN treatment, some normal sieve plates were maintained however there was a significant reduction of cytoplasmic area between sieve plates resulting in a hyper-fenestrated morphology, similar to the effects seen with 7-ketocholesterol in this study. 7-ketocholesterol was associated with increased fenestration diameter in both young and old mice (P<0.05; FIG. 12A.)

There were effects of the drugs on the frequency distribution of fenestration diameter. NMN was associated with smaller fenestrations (less than 75 nm) on the edge of sieve plates (FIGS. 12B and 12C) in young mice but not old mice. In young mice, NMN (5000 μg/ml) induced an increase in 30-100 nm and 226-500 nm fenestrations with a reduction in 126-200 nm fenestrations (FIG. 2C). In older mice, NMN treatment shifted the diameter of fenestrations from a peak of 76-100 nm to 101-125 nm and was associated with a reduction in smaller fenestrations (diameter 30-100 nm) (FIG. 12C). This effect was not observed with 7-ketocholesterol (3.6 μg/ml) treatment, instead a shift to the right with decreased 30-125 nm diameter fenestrations and an increase in 150-3000 sized fenestrations occurred in young mice. In old mice, 7-ketocholesterol (3.6 μg/ml) demonstrated a peak at 101-125 nm with increased 150-300 nm fenestrations similarly to NMN.

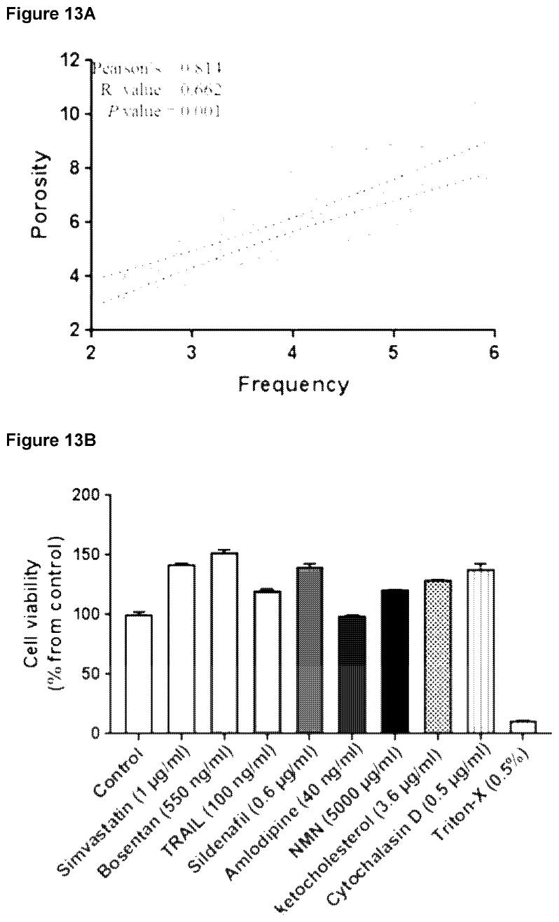

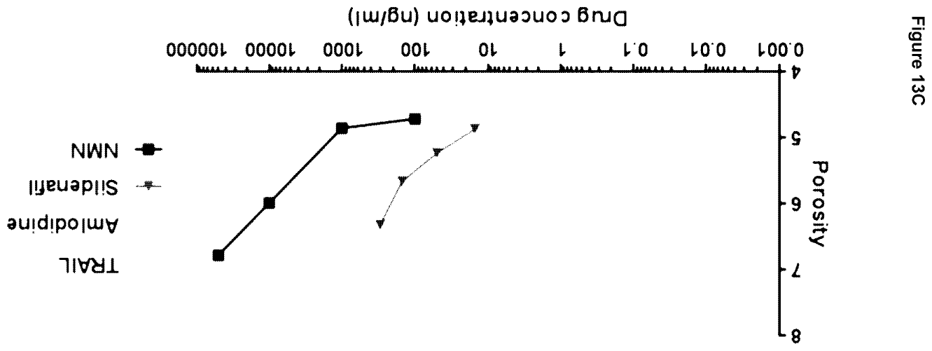

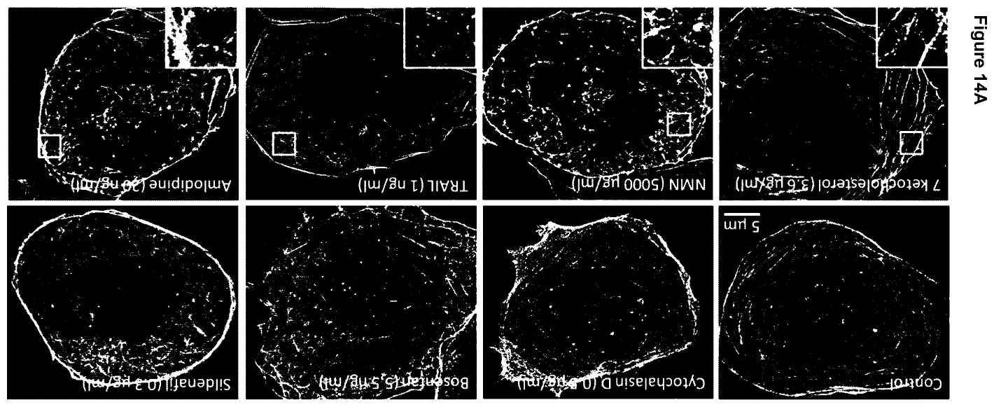

Porosity was primarily increased as a result of increased numbers rather than the size of fenestrations (FIG. 13A). Cell viability was assessed via an MTT assay and demonstrated maximal drug dosages did not induce cellular toxicity (FIG. 13B). Dose response experiments were performed in young mice for all drugs that were shown to be active in modulating fenestration porosity (FIG. 13C). TRAIL had the greatest activity and similar maximal efficacy to NMN but was more potent (FIG. 13C). Sildenafil, amlodipine and TRAIL however, had a limited dosage range for positive effects on fenestration porosity while NMN had a broad range. NMN treatment resulted in the largest increase in fenestration porosity from 4.6% to 8.1% in LSECs from young mice.

### Effects of Agents on Actin and Nitric Oxide Synthase

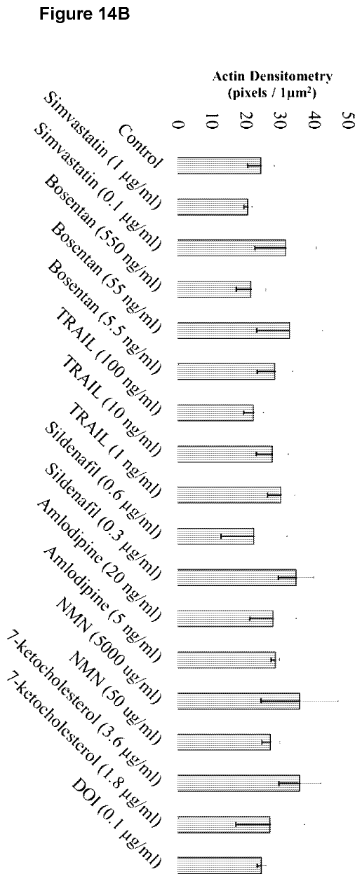

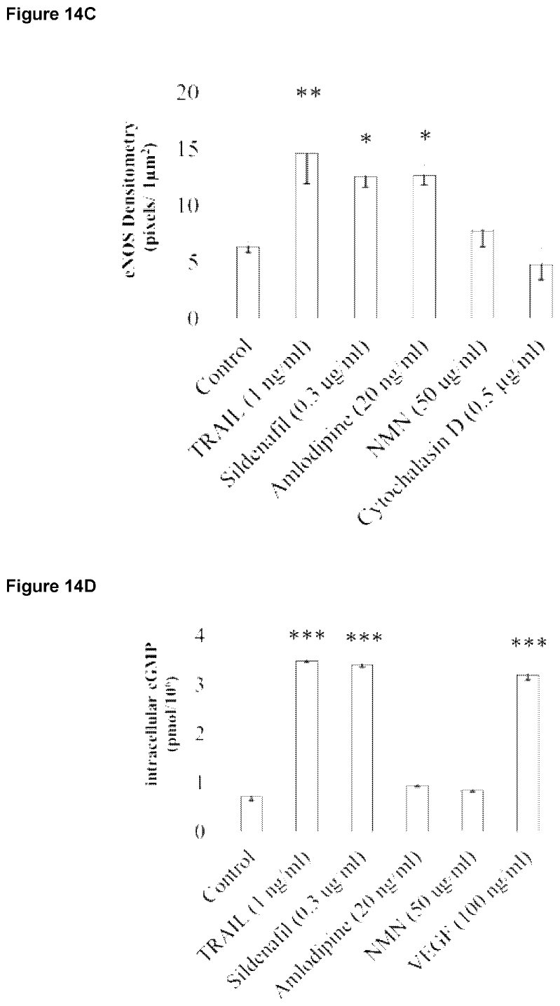

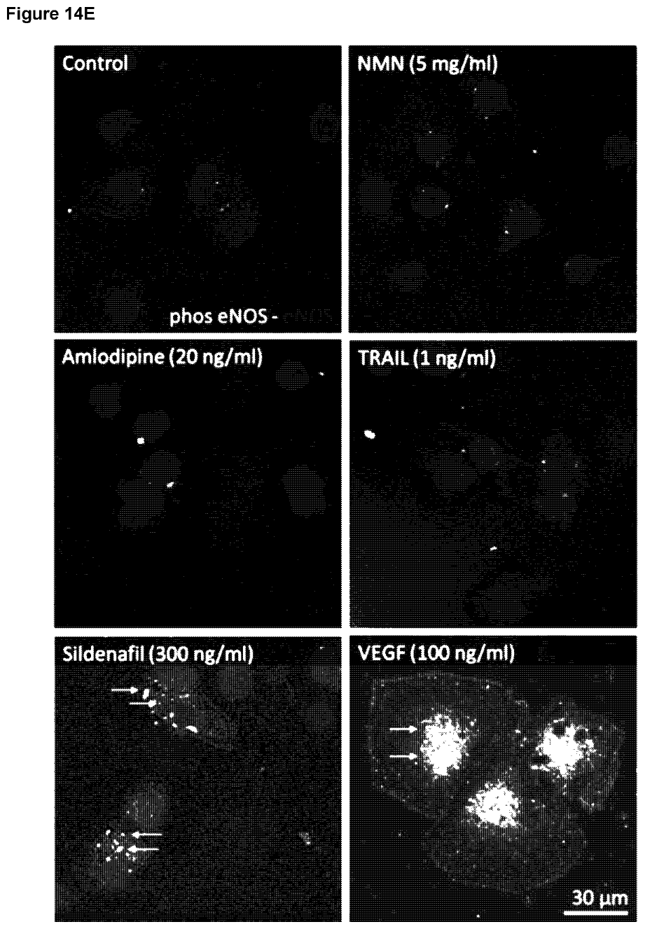

Control LSECs demonstrated moderate actin staining within the plasma membrane and cytoplasm including broad circular tubular structures (FIG. 14A). No changes in actin density in LSECs were observed (FIG. 14B). Changes in the pattern of actin cyto-architecture were observed between treatment groups (Table 4) while the overall quantity of actin in the cells was unchanged.

TABLE 4
Actin and nitric oxide synthase changes with drug treatments

| Cytochalasin D | Amlodipine and NMN |
|---|---|
| Disordered actin structure | Disordered actin structure |
| Dense actin plasma membrane | Dense actin plasma membrane |
| Stress fibres | Actin clusters |
| Single isolated fenestrations | Gap formations |
| No NOS or pNOS changes | Individual fenestrations |
| | Increased NOS in amlodipine |
| Sildenafil | TRAIL |
| Disordered actin structure | Disordered actin structure |
| Dense actin plasma membrane | Gap formations |
| Gap formations | Minimal actin clustering |
| Individual fenestrations | Increased NOS |
| Increased NOS and pNOS | |
| Bosentan | 7-ketocholesterol |
| Stress fibres | Ordered actin structure |
| Fused actin structure | Extensive gap formations |
| Individual fenestrations | within the cytoplasm |

LSECs treated with cytochalasin D had extensive actin staining of the plasma membrane (FIG. 14A). Stress fibres were present within the peri-nuclear area. There was a loss of smooth fibres encircling the cytoplasm following treatment with cytochalasin D, amlodipine, NMN and sildenafil.

Sildenafil, amlodipine and NMN demonstrated a similar phenotype with disordered, dense actin staining in the plasma membrane and clustering of actin within the cytoplasm (FIG. 14A). The key features were: (1) fibres projected in all directions, (2) actin clusters, (3) gap formation, and (4) individual fenestrations visible in some sieve plates (FIG. 14A inserts). TRAIL was similar to sildenafil, amlodipine and NMN apart from absence of the intense actin clustering (Table 4, FIG. 14A).

7-ketocholesterol treatment was associated with organised actin structure throughout the cytoplasm similar to controls (FIG. 14A). However, large gaps occurred throughout the actin cyto-architecture, actin fibres maintained their continuous and interconnected appearance but lost their circular tubular structures. Moderate staining was seen in the plasma membrane. The large gaps were also observed in the cytoplasmic actin (FIG. 14A, insert).

Changes in the actin cytoskeleton were associated with increased fenestration porosity and frequency; however, there didn't appear to be any specific pattern of change in the cytoskeleton that was associated with increased fenestrations with all treatments.

Increased NOS densitometry was observed for TRAIL, amlodipine and sildenafil (FIG. 14C). Intracellular cGMP was increased 3-fold following sildenafil and TRAIL treatments (p=0.001); no changes were observed in NMN or amlodipine treated cells (FIG. 14D). Control LSECs and those treated with NMN demonstrated minimal NOS staining and non-phosphorylated NOS (FIG. 14E). TRAIL and amlodipine showed NOS staining across the cytoplasm but without phosphorylated NOS (FIG. 14E). Sildenafil and VEGF (100 ng/ml, 4 hr treatment) showed staining for both NOS and phosphorylated NOS (FIG. 14E, white arrows).

### Discussion

The morphology of fenestrations in LSECs is responsive to a variety of pharmacological interventions and this responsiveness is mostly maintained into older age. LSECs isolated from old mice in this study had reduced porosity and frequency of fenestrations, consistent with previous studies in mice as well as rats, humans and non-human primates. NMN, sildenafil and 7-ketocholesterol increased fenestration porosity and frequency in young mice, with similar or greater effects seen in LSECs from old mice (summary data provided in Table 5). This indicates that age-related defenestration can be reversed in vitro and may be a valid therapeutic target for in vivo studies. Moreover, the optimal concentrations of these refenestrating agents were identified in LSECs from old mice, providing a potential target dose for in vivo studies. The results of the dSTORM studies showed that refenestration was associated with significant actin reorganization. Increased NOS protein expression was also seen in LSECs treated with amlodipine, sildenafil, and TRAIL while sildenafil was the only agent associated with increased phosphorylation of NOS. Overall, our study indicates that agents that increased fenestrations are associated with an alteration of the actin cytoskeleton and in some cases, release of NO; importantly this responsiveness is maintained in old age.

TABLE 5
Changes in fenestration porosity, diameter and
frequency promoted by various drug and agents.

| | Porosity | | Diameter | | Frequency | |
|---|---|---|---|---|---|---|
| Drug | Young | Old | Young | Old | Young | Old |
| Simvastatin (1 μg/ml) | — | — | — | ↑ (ns) | — | — |
| Simvastatin (0.1 μg/ml) | — | — | — | ↑ (ns) | — | — |
| Bosentan (550 ng/ml) | — | — | — | — | — | — |
| Bosentan (55 ng/ml) | — | — | — | — | — | — |
| Bosentan (5.5 ng/ml) | — | ↑ | — | — | — | ↑ |
| TRAIL (100 ng/ml) | — | — | — | — | — | — |
| TRAIL (10 ng/ml) | ↑ (ns) | — | — | — | — | — |
| TRAIL (1 ng/ml) | ↑ | — | — | — | ↑ | — |
| Sildenafil (0.6 μg/ml) | — | ↑ | — | — | — | — |
| Sildenafil (0.3 μg/ml) | ↑ | ↑ | — | — | ↑ | ↑ |
| Amlodipine (20 ng/ml) | ↑ | ↑ (ns) | — | — | ↑ | ↑ (ns) |
| Amlodipine (5 ng/ml) | — | ↑ | — | — | — | — |
| NMN (5000 μg/ml) | ↑ | ↑ | — | — | ↑ | ↑ |
| NMN (50 μg/ml) | — | ↑ | — | — | ↑ | ↑ |
| 7-ketocholesterol (3.6 μg/ml) | ↑ | ↑ | ↑ | — | ↑ | ↑ |
| 7-ketocholesterol (1.8 μg/ml) | — | ↑ | — | ↑ | — | — |
| DOI (1 μg/ml) | — | — | — | ↑ | — | — |
| DOI (0.1 μg/ml) | — | ↑ | — | — | — | ↑ (ns) |
| Cytochalasin D (0.5 μg/ml) | ↑ (ns) | | | | ↑ | ↑ |

↑ = increased (P < 0.05); (ns) = (P < 0.1).

In old mice, NMN (50 μg/ml) generated the greatest increase in fenestration porosity and frequency. NMN is a biosynthetic nicotinamide adenine dinucleotide (NAD+) metabolite that is critical for the regulation of NAD+ biosynthesis via the NAD+ salvage pathway. NMN is converted to NAD+ by NMN acetyltransferase and is produced from the NAD+ breakdown product nicotinamide in the presence of nicotinamide phosphoribosyltransferase. This salvage process occurs in the nucleus, mitochondria and cytosol and maintains high levels of NAD+ in the liver. Elevated NAD+ is promoted 15 mins following a single intraperitoneal injection of 500 mg/kg NMN in female mice. In old rats, it has been shown that this dosage is non-toxic and promotes improved glucose tolerance. Similar dosages given continuously for 7 days were also shown to improve insulin action and secretion in diet and age induced type 2 diabetic mice models. The data presented herein suggest that one mechanism for the effects of NMN on glucose/insulin metabolism might involve refenestration of the old LSEC leading to increased insulin sensitivity in the liver. In young LSECs, NMN (5000 μg/ml concentration) generated increased fenestration porosity and frequency with shifts in the distribution of diameter. The fenestration diameter histogram (FIG. 12C) highlights the presence of small 30-100 nm fenestrations and larger 125-300 fenestrations following 30 min of treatment. NMN increased the frequency of fenestrations substantially which suggests that the increase in the proportion of small fenestrations might represent the formation of new fenestrations. In old mice, NMN treatment shifted the diameter of fenestrations to the right with an increase in fenestration diameter. Consequently, the average fenestration diameter in old mice treated with NMN was similar to young control mice (old NMN: 132±2 nm vs young control: 131±7 nm).

These agents also had varying effects on the actin cytoskeleton as visualized using dSTORM. The condensation and clustering of actin appeared to be similar following treatment with cytochalasin D, amlodipine, sildenafil and NMN. However, treatment with 7-ketocholesterol produced a diffuse and stretched actin network, possibly generated by the retraction of lipid rafts that are anchored to the actin cytoskeleton and this was associated with a significant 15 nm increase in fenestration diameter. This suggests that agents that act upstream on the actin cytoskeleton will largely influence frequency of fenestrations and agents that act directly on lipid rafts may additionally increase the diameter of fenestrations, perhaps as a result of increased non-lipid raft cell membrane.

The regulation of LSEC fenestrations has been recently reviewed and the major regulatory pathway is thought to be mediated by VEGF and NO. Three drugs that influence NOS and NO were investigated: amlodipine, sildenafil and simvastatin. Only sildenafil influenced LSECs in both young and old mice, amlodipine showed a similar pattern in fenestration changes but did not demonstrate statistical significance. Sildenafil promotes cGMP and PKG via inhibition of PKES leading to increase NO availability. Amlodipine has a dual action on NO via cGMP and inhibition of Ca2+ channels. Sildenafil does not inhibit Ca²⁺ influx. Simvastatin promotes the releases of NO from the endothelium via an Akt-dependent pathway and inhibits Rho GTP-kinase to indirectly promote cGMP and PKG activation. Simvastatin does not promote Ca²⁺ flux. This study showed that sildenafil, and to a weaker extent amlodipine, promoted changes in fenestration porosity and frequency, with increased NOS expression. Simvastatin in comparison promoted a non-significant increase in fenestration diameter. These findings support the NO-cGMP-PKG pathway proposed but suggest that direct targeting of cGMP and PKG signalling (such as by sildenafil and amlodipine) may promote greater fenestration porosity and frequency and targeting Akt-dependent NO release via simvastatin may increase fenestration diameter. Future studies are required to determine whether these drugs increase fenestrations in old animals in vivo, and whether this leads to increased hepatic clearance of circulating insulin and lipoproteins.

The effects of TRAIL were also invetsigated. TRAIL is a death receptor agonist and promotes caspase-8 dependant programed cell death. In old mice, TRAIL had minimal effects on the LSEC however in young mice; TRAIL was associated with a 60% increase in porosity and a 40% increase in fenestration frequency. TRAIL had similar effects as sildenafil in terms of effects on fenestration frequency and diameter, actin and NOS. TRAIL has been reported to upregulate NOS and phosphorylated NOS following 15 mins of 1 μg/ml treatment in human umbilical vein endothelial cells. Together, these results indicate that amongst its other established effects, TRAIL also influences NOS expression in endothelial cells.

Previously it has been reported that cytochalasin D, 7-ketocholesterol and DOI increase fenestration porosity in young mice without any significant effects on fenestration diameter. In the recent studies we observed increased porosity with 7-ketocholesterol only, however cytochalasin D demonstrated a 33% increase but was not significant (P=0.08). We also found that cytochalasin D, 7-ketocholesterol but not DOI increased fenestrations in LSECs from old mice. However, we previously reported that in vivo administration of DOI increased fenestrations only in young (7 month) but not old (24 month) mice. The difference in these results presumably reflects the different methodologies (in vivo vs in vitro) and ages (18 month vs 24 month) used in these studies.

In conclusion, the present inventors have shown that in vitro drug treatments with NMN, sildenafil and 7-ketocholesterol increase fenestration porosity and frequency in LSECs isolated from young and old mice. The regulation of fenestrations may be mediated by NO-dependent and -independent pathways. Defenestration associated with age-related pseudocapillarization can be reversed by several different agents, which may have an impact on age-related dyslipidaemia and hepatic insulin resistance.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Although the invention has been described with reference to a preferred embodiment, it will be appreciated by persons skilled in the art that the invention may be embodied in many other forms. It will be appreciated by persons skilled in the art that numerous variations and/or modifications may be made to the technology as shown in the specific embodiments without departing from the spirit or scope of technology as broadly described. The present embodiments are, therefore, to be considered in all respects as illustrative and not restrictive.

1. A composition for modulating one or more of endothelial cell fenestration porosity, diameter and frequency in a subject, the composition comprising a therapeutic conjugate comprising a quantum dot and a therapeutic selected from an endothelin receptor antagonist, phosphodiesterase (PDE) inhibitor, calcium channel blocker, actin disruptor, lipid raft disruptor, 5-HT receptor agonist, TNF-related apoptosis-inducing ligand (TRAIL), nicotinamide adenine mononucleotide (NMN) or a combination thereof.

2. The composition of claim 1 wherein the quantum dot is an Ag₂S, InP/ZnS or CuInS/ZnS quantum dot.

3. The composition of claim 1 wherein the subject is an aged subject or a subject with an age related disease or condition.

4. The composition of claim 1 wherein the average diameter of the quantum dot is about 2 nm, 3 nm, 4 nm, 5 nm, 6 nm, 7 nm, 8 mn, 9 nm, 10 nm, 11 nm, 12 nm, 13 nm, 14 nm, 15 nm, 16 nm, 17 nm, 18 nm or 20 nm.

5. The composition of claim 1 wherein the therapeutic conjugate is monodispersed.

6. The composition of claim 1 wherein the endothelin receptor antagonist is selected from bosentan, sitaxentan, ambrisentan, atrasentan, zibotentan, macitentan, tezosentan, and edonentan.

7. The composition of claim 1 wherein the phosphodiesterase (PDE) inhibitor is selected from sildenafil or its active analogues, tadalafil, vardenafil, udenafil, and avanafil.

8. The composition of claim 1 wherein the calcium channel blocker is selected from amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine, fendiline. In another embodiment the calcium channel blocker is amlodipine.

9. The composition of claim 1 wherein the actin disruptor is selected from cytochalasin, latrunculin, jasplakinolid, phalloidin, and swinholide.

10. The composition of claim 1 wherein the lipid raft disruptor is selected from filipin, 7-ketocholesterol (7KC), and methyl-β-cyclodextrin.

11. The composition of claim 1 wherein the 5-HT receptor agonist is selected from 2,5-Dimethoxy-4-iodoamphetamine (DOI), haloperidol, aripiprazole, asenapine, buspirone, vortioxetine, ziprasidone, methylphenidate, dihydroergotamine, ergotamine, methysergide, almotriptan, eletriptan, frovatriptan, naratriptan, rizatriptan, sumatriptan, zolmitriptan, yohimbine, lasmiditan, naratriptan, bufotenin, egonovine, lisuride, LSD, mescaline, myristicin, psilocin, psilocybin, fenfluramine, MDMA, norfenfluramine, methylphenidate, ergonovine, lorcaserin, tazodone, methyl-5-HT, qipazine, cinitapride, cisapride, dazopride, metoclopramide, mosapride, prucalopride, renzapride, tegaserod, zacopride, ergotamine, and valerenic acid.

12. A method of modulating one or more of endothelial cell fenestration, porosity, diameter and frequency in a subject, the method comprising administering to the subject an effective amount of a composition of claim 1.

13. The method of claim 12 wherein the subject is a subject with an age related disease or condition.

14. The method of claim 12 wherein the age related disease or condition is selected from atherosclerosis, cardiovascular disease, arthritis, cataracts, age-related macular degeneration, hearing loss, osteoporosis, osteoarthritis, type 2 diabetes, hypertension, Parkinson's disease, dementia, Alzheimer's disease, age-related changes in the liver microcirculation, age-related dyslipidaemia, insulin resistance, fatty liver, liver fibrosis and liver cirrhosis.

15. The method of claim 12 wherein the subject is a subject with a disease or condition associated with one or more of reduced endothelial cell fenestration porosity, diameter and frequency.

16. The method of claim 12 wherein the therapeutic or therapeutic conjugate associates with an endothelial cell.

17. The method of claim 12 wherein the therapeutic conjugate selectively associates with an endothelial cells.

18. The method of claim 16 wherein the endothelial cell is a liver endothelial cell.

19. The method of claim 12 wherein the modulation is an increase in one or more of endothelial cell fenestration porosity, diameter and frequency.

20. The method of claim 19 wherein the increase is at least 5%.

21. (canceled)

22. A method of modulating one or more of endothelial cell fenestration porosity, diameter and frequency in a subject, the method comprising administering to the subject an effective amount of a phosphodiesterase (PDE) inhibitor, calcium channel blocker, actin disruptor, lipid raft disruptor, 5-HT receptor agonist, TNF-related apoptosis-inducing ligand (TRAIL), nicotinamide adenine mononucleotide (NMN) or a combination thereof.

23. The method of claim 22 wherein the endothelin receptor antagonist is selected from bosentan, sitaxentan, ambrisentan, atrasentan, zibotentan, macitentan, tezosentan, and edonentan.

24. The method of claim 23 wherein the phosphodiesterase (PDE) inhibitor is selected from sildenafil or its active analogues, tadalafil, vardenafil, udenafil, and avanafil.

25. The method of claim 23 wherein the calcium channel blocker is selected from amlodipine, aranidipine, azelnidipine, barnidipine, benidipine, cilnidipine, clevidipine, efonidipine, felodipine, isradipine, lacidipine, lercanidipine, manidipine, nicardipine, nifedipine, nilvadipine, nimodipine, nisoldipine, nitrendipine, pranidipine, fendiline. In another embodiment the calcium channel blocker is amlodipine.

26. The method of claim 23 wherein the actin disruptor is selected from cytochalasin, latrunculin, jasplakinolid, phalloidin, and swinholide.

27. The method of claim 23 wherein the lipid raft disruptor is selected from filipin, 7-ketocholesterol (7KC), and methyl-β-cyclodextrin.

28. The method of claim 27 wherein the 5-HT receptor agonist is selected from 2,5-Dimethoxy-4-iodoamphetamine (DOI), haloperidol, aripiprazole, asenapine, buspirone, vortioxetine, ziprasidone, methylphenidate, dihydroergotamine, ergotamine, methysergide, almotriptan, eletriptan, frovatriptan, naratriptan, rizatriptan, sumatriptan, zolmitriptan, yohimbine, lasmiditan, naratriptan, bufotenin, egonovine, lisuride, LSD, mescaline, myristicin, psilocin, psilocybin, fenfluramine, MDMA, norfenfluramine, methylphenidate, ergonovine, lorcaserin, tazodone, methyl-5-HT, qipazine, cinitapride, cisapride, dazopride, metoclopramide, mosapride, prucalopride, renzapride, tegaserod, zacopride, ergotamine, and valerenic acid.

29. (canceled)
